(19) United States
(12) Patent Application Publication
(10) Pub. No.: US 2022/0378751 A1
(43) Pub. Date: Dec. 1, 2022

(54) INHIBITING DIHYDROFOLATE REDUCTASE IN A MICROORGANISM

(71) Applicant: VERSITECH LIMITED, Hong Kong (HK)

(72) Inventors: Yi Tsun Richard KAO, Hong Kong (HK); Hin Cheung Bill YAM, Hong Kong (HK); Peng GAO, Hong Kong (HK); Ho Ting IU, Hong Kong (HK)

(21) Appl. No.: 17/870,855

(22) Filed: Jul. 22, 2022

Related U.S. Application Data
(63) Continuation-in-part of application No. 16/309,516, filed on Dec. 13, 2018.
(60) Provisional application No. 62/349,823, filed on Jun. 14, 2016.

(51) Int. Cl. A61K 31/426; A61K 31/427; A61K 31/7008; A61K 31/357; A61P 31/04

(52) U.S. Cl. CPC A61K 31/426; A61K 31/427; A61K 31/7008; A61K 31/357; A61P 31/04

(57) ABSTRACT

The present invention can inhibit dihydrofolate reductase in a microorganism. In some aspects, it may also suppress pigment formation in gram negative bacteria. The suppression of pigment formation can also mediate virulence suppression on other Gram negative bacteria. As such, the compounds may be used for microbial infections. The compounds can potentiate the effects of one or more antimicrobial agents when co-administered. Compositions including the compounds are provided. The composition can further include at least one antibiotic.

### CROSS REFERENCE TO RELATED APPLICATION

This application is a continuation-in-part of U.S. patent application Ser. No. 16/309,516 filed Dec. 13, 2018, which is a national phase entry of international patent application No. PCT/CN2017/087898 filed on Jun. 12, 2017 which claims priority to U.S. Provisional Patent Application No. 62/349,823 filed on Jun. 14, 2016; the disclosures of which are incorporated herein by reference in their entirety.

### FIELD OF THE INVENTION

The invention is generally directed to compounds for inhibiting dihydrofolate reductase in a microorganism.

### BACKGROUND OF THE INVENTION

Infectious diseases have been the prime cause of death ever since human population existed, and bacterial infection plays a major role in this medical challenge. Nosocomial infection and antibiotic-resistant bacteria related infection has become the major challenge in the medical field for past decades. Attentions were focused on to the ESKAPE pathogens (acronymically for the Enterococcus faecium, Staphylococcus aureus, Klebsiella pneumoniae, Acinetobacter baumannii, Pseudomonas aeruginosa, and Enterobacter species), their well adaptations in virulence for infections or multidrug resistances for treatment withstanding made therapeutic options very limiting (Khan et al., Front Microbiol., 7:174 (2016)). Rapid developments of resistance mechanisms render new drugs less effective in a short time following use. The shrinking of the pharmaceutical antibiotics pipeline due to the low profit margins only makes this situation worse (Butler et al., The Journal of antibiotics, 66(10):571-591 (2013)). It is foreseeable that in the coming future, more resistant bacteria will emerge, and the lack of potent antimicrobials against bacteria will become a major threat (Tommasi et al., Nat Rev Drug Discov., 14(8): 529-42 (2015)). New antimicrobials/treatment methods against the bacteria are therefore needed.

Antibiotic resistance is a major problem for bacterial infections. Efforts have been devoted to the development of new compounds for bacterial growth inhibition. However, no major achievements have been made in the past decades. Current treatment options for antibiotics resistant bacteria still rely on combinatory effect of several second or third line antibiotics. There is still a need for better treatment options.

It is therefore an object of the present invention to provide compounds with antimicrobial activity.

It is also an object of the present invention to provide compounds that potentiate the antimicrobial effect of existing antibiotics.

It is a further object of the present invention to provide compositions for treating bacterial infections.

It is still an object of the present invention to provide methods for treating a subject with a microbial infection.

It is also an object of the present invention to provide compounds with antibacterial activity, which can be used to combat drug resistant bacterial infections.

### SUMMARY OF THE INVENTION

Disclosed are compounds, compositions, and methods for, for example, treating microbial infections, disinfecting surfaces, increasing the efficacy of antibiotics, and reducing virulence of bacteria. Some forms provide compounds with antimicrobial properties including compounds 2-4, 6-7, 10, 13, 15, 17-20, 22, 24-25, 27-31, 34-37, 42, 44, 48-50, 52-53, 56, 60, 62-63, 69, 80-82 and 87 as further disclosed below. In some forms, the compound potentiates the therapeutic effects of one or more antimicrobial agents when coadministered. In the other forms the compounds have antivirulence properties, measured as repression in hla promoter lux reporter assay and suppression of pigment production.

Pharmaceutical compositions including the compounds disclosed herein in a pharmaceutically acceptable carrier are provided. Preferred compounds include compounds 2-4, 6-7, 10, 13, 15, 17-20, 22, 24-25, 27-31, 34-37, 42, 44, 48-50, 52-53, 56, 60, 62-63, 69, 80-82 and 87. In this form, the pharmaceutical composition may further comprise at least one antibiotic, such as, Gentamicin, amikacin, kanamycin, neomycin, spectinomycin, neamine or any combination thereof.

The compounds can be formulated into cleaning formulations. The cleaning formulations are highly efficacious for household cleaning applications (e.g., hard surfaces like floors, countertops, tubs, dishes and softer cloth materials like clothing, sponges, paper towels, etc.), personal care applications (e.g., lotions, shower gels, soaps, shampoos, wipes) and industrial and hospital applications (e.g., sterilization of instruments, medical devices, gloves).

Also disclosed are methods of treating or preventing microbial infection, resulting from microbes such Staphylococcus aureus, Pseudomonas aeruginosa, Listeria monocytogenes, Burkholderia cepacia, Escherichia coli, Enterococcus faecalis, Streptococcus pneumoniae or disinfecting a surface contaminate with microbes. The method includes administering an effective amount of one or more compound according to any of formulas 2-4, 6-7, 10, 13, 15, 17-20, 22, 24-25, 27-31, 34-37, 42, 44, 48-50, 52-53, 56, 60, 62-63, 69, 80-82 and 87 to a subject in need thereof. In some forms, the methods further comprise administering an effective amount of at least one antibiotic, such as, e.g., gentamicin, amikacin, kanamycin, neomycin, spectinomycin, neamine or any combination thereof.

The antibiotics may be administered separately, simultaneously or sequentially. In a preferred form, the amount of a separately, simultaneously, or sequentially administered one or more antimicrobial agents (including one or more antibiotics) may be less than a therapeutically effective or therapeutically optimal dose of the one or more antimicrobial agents (including one or more antibiotics) when administered to the patient or animal that is not in receipt of the disclosed compounds.

Disclosed are compounds represented by Formula I

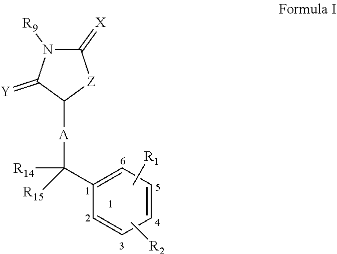

where X and Y are independently O, S, or NR₁₀; where Z is O, S, CR₁₁R₁₂, or NR₁₃; where R₁, R₂, R₉, R₁₀, R₁₁, R₁₂, R₁₃, R₁₄, and R₁₅, are independently absent,

hydrogen, —C(O)R₁₈, —C(W)NR₁₉R₂₀, or substituted or unsubstituted alkyl, alkenyl, alkynyl, alkoxy, cycloalkyl, heterocyclyl, cycloalkenyl, heterocycloalkenyl, aryl, heteroaryl, aroxy, arylalkyl, heteroalkyl, alkylaryl, halogen, alkylheteroaryl, —NR₁₆R₁₇; wherein R₁₆ and R₁₇ are independently hydrogen, substituted or unsubstituted alkyl, alkenyl, alkynyl, cycloalkyl, heterocyclyl, cycloalkenyl, heterocycloalkenyl, aryl, heteroaryl, arylalkyl, heteroalkyl, alkylaryl, or alkylheteroaryl;

where R₁₈ is hydrogen, hydroxyl, or substituted or unsubstituted alkyl, alkenyl, alkynyl, cycloalkyl, alkoxy, heterocyclyl, cycloalkenyl, heterocycloalkenyl, aryl, aroxy, heteroaryl, arylalkyl, heteroalkyl, alkylaryl, or alkylheteroaryl;

where W is O, S, or NR₂₁;

where R₁₉, R₂₀, and R₂₁ are independently hydrogen or substituted or unsubstituted alkyl, alkenyl, alkynyl, cycloalkyl, heterocyclyl, cycloalkenyl, heterocycloalkenyl, aryl, heteroaryl, arylalkyl, heteroalkyl, alkylaryl, or alkylheteroaryl;

where A is a double bond or single bond; and

where R₁₅ is absent when A is a double bond.

In some forms, the compound can be

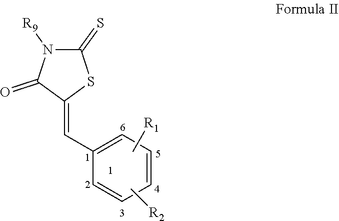

where R₁ is —O—R₄, —NR₅R₈, or —CH₂—R₅; R₂ is halogen, absent, —O—CH₃, or —O—CH₂—CH₃; R₉ is —COOH, —CH₂—COOH, —CH₂—O—CH₃, or —CH₂—CH₂—O—CH₃; R₄ is —CH₂—R₅ or —CO—R₅; R₅ is

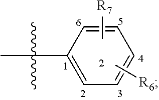

R₆ is a halogen or absent; and R₇ is halogen or absent.

In some forms of the compound, R₁ is at a position selected from the group consisting of 2, 3, 4, 5, and 6. In some forms, R₆ is at position 4. In some forms, R₇ is at position 2 or 6. In some forms, R₂ is at position 3, 4, or 5. In some forms, R₆ is at position 4, R₇ is at position 2 or 6, and R₂ is at position 3 or 5.

In some forms, the compound is

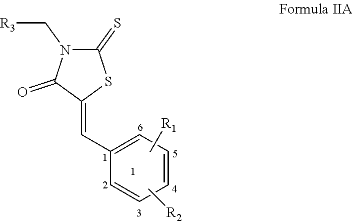

where R₁ is —O—R₄, NR₅R₈, or —CH₂—R₅; R₂ is halogen absent, —O—CH₃, or —O—CH₂—CH₃; R₃ is —COOH or —CH₂—COOH; R₄ is —CH₂—R₅ or —CO—R₅; R₅ is

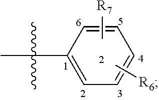

R₆ is halogen or absent; and R₇ is halogen or absent.

In some forms, R₁ is at a position selected from the group consisting of 2, 3, 4, 5, and 6. In some forms, R₆ is at position 4. In some forms, R₇ is at position 2 or 6. In some forms, R₂ is at position 3, 4, or 5. In some forms, R₆ is at position 4, R₇ is at position 2 or 6, and R₂ is at position 3 or 5.

In some forms, the compound is selected from the group consisting of compounds 2-4, 6-7, 10, 13, 15, 17-20, 22, 24-25, 27-31, 34-37, 42, 44, 48-50, 52-53, 56, 60, 62-63, 69, 80-82, and 87.

Also disclosed are compositions comprising an effective amount of one or more of the disclosed compounds and a pharmaceutically acceptable carrier. Such compositions are useful for, for example, treating microbial infections in a subject. In some forms, the effective amount of the compound is effective to inhibit dihydrofolate reductase in a microorganism.

In some forms, the composition further comprises a therapeutically effective amount of an antibiotic. In some forms, the antibiotic is selected from the group consisting of gentamicin, amikacin, kanamycin, neomycin, spectinomycin, neamine and combinations thereof. In some forms, the therapeutically effective amount of the antibiotic is less than a therapeutically effective or therapeutically optimal dose of the antibiotic when administered to a subject in the absence of the composition.

Also disclosed is a method of treating a microbial infection in a subject, the method comprising administering to the subject one or more of the compositions. In some forms, the microbial infection is caused by one or more microorganisms and the composition is effective to inhibit dihydrofolate reductase in at least one of the microorganisms causing the infection.

In some forms, at least one of the microorganisms causing the infection is bacteria selected from the group consisting of methicillin-susceptible Staphyloccous aureus, Pseudomonas aeruginosa, Listeria monocytogenes, Burkholderia cepacia, Escherichia coli, Enterococcus faecalis, Streptococcus pneumoniae or combinations thereof.

In some forms, at least one of the microorganisms causing the infection is an antibiotic-resistant microorganism selected from the group consisting of a Streptococcus pneumoniae, Campylobacter, Neisseria gonorrhoeae, Salmonella, Methicillin-resistant Staphylococcus aureus (MRSA), Shigella, Vancomycin-resistant Enterococcus (VRE), Vancomycin-resistant Staphylococcus aureus (VRSA), Erythromycin-resistant Group A Streptococcus, Clindamycin-resistant Group B Streptococcus, Carbapenem-resistant Enterobacteriaceae (CRE), drug-resistant tuberculosis, Extended spectrum Enterobacteriaceae (ESBL), multidrug-resistant Acinetobacter (including MRAB), Clostridium difficile, Enteropathogenic E. coli (EPEC), Pseudomonas aeruginosa, H. pylori, Streptococcus anginosus and Uropathogenic E. coli (UPEC).

In some forms, at least one of the microorganisms causing the infection is Methicillin-resistant Staphylococcus aureus (MRSA).

In some forms, the microorganism causing the infection is a multi-drug-resistant strain of Listeria monocytogenes, Pseudomonas aeruginosa, Burkholderia cepacia, Enterococcus faecalis and Streptococcus pneumoniae.

In some forms, the infection is selected from the group consisting of impetigo, boils, abscesses, folliculitis, cellulitis, necrotizing fasciitis, pyomyositis, surgical/traumatic wound infection, and infected ulcers and burns), osteomyelitis, device-related osteoarticular infections, impetigo, secondarily infected skin lesions, meningitis, brain abscess, subdural empyema, spinal epidural abscess.

In some forms, the infection is a urinary tract infection. In some forms, the composition is administered parenterally or orally. In some forms, the subject is hospitalized or immunocompromised.

Also disclosed is a method of disinfecting a surface in need thereof, the method comprising contacting the surface with one or more of the disclosed compounds or with one or more of the disclosed compositions.

In some forms, the compound is applied on the surface in the form of a spray, an aerosol, or a foam. In some forms, the compound is imbibed into a cloth suitable for wiping down a surface to be disinfected. In some forms, the surface is the surface of an instrument selected from the group consisting of surgical instruments, cardiac and urinary catheters, implants, and ultrasound probes used in sterile body cavities.

In some forms, the instrument is selected from the group consisting of endoscopes, laryngoscope blades, esophageal manometry probes, cystoscopes, anorectal manometry catheters, diaphragm fitting rings, gastroscopes, duodenoscopes, sigmoidoscopes, proctoscopes, colonoscopes, bronchoscopes, and laryngoscopes.

Also disclosed is a method of increasing the efficacy of an antibiotic comprising co-administering the antibiotic and one or more of the disclosed compounds or one or more of the disclosed compositions in amount effective to increase the efficacy of the antibiotic.

In some forms, the compound or composition is administered separately, simultaneously, or sequentially with the antibiotic. In some forms, efficacy of the antibiotic is increased by at least 2 fold measured as inhibition of growth of a microorganism in an in vitro assay in response to the antibiotic alone, compared to growth inhibition of the microorganism in response to a combination the antibiotic and the compound or composition.

Also disclosed is a method of reducing virulence of bacteria selected from the group consisting of methicillin-resistant Staphyloccous aureus, Pseudomonas aeruginosa, Listeria monocytogenes, Burkholderia cepacia, Escherichia coli, Enterococcus faecalis, Streptococcus pneumoniae or combinations thereof in a subject comprising administering to the subject an effective amount of one or more of the disclosed compounds or one or more of the disclosed compositions.

In some forms, the compound is selected from the group consisting of compounds 3, 48, 49, and 87.

### BRIEF DESCRIPTION OF THE DRAWINGS

FIG. 1 shows a microdilution checkerboard analysis showing the combined effect of compound 13 with gentamicin. The analysis shows that compound 13 has potentiating effect with gentamicin in inhibiting the growth of S. aureus Mu3 strain. Heat Plot shows the average growth from replicated experiments.

FIG. 2 shows a microdilution checkerboard analysis showing the combined effect of compound 13 with amikacin. The analysis shows that compound 13 has potentiating effect with amikacin in inhibiting the growth of S. aureus Mu3 strain. Heat Plot shows the average growth from replicated experiments.

FIG. 3 shows a microdilution checkerboard analysis showing the combined effect of compound 13 with kanamycin. The analysis shows that compound 13 has potentiating effect with kanamycin in inhibiting the growth of S. aureus Mu3 strain. Heat Plot shows the average growth from replicated experiments.

FIG. 4 shows a microdilution checkerboard analysis showing the combined effect of compound 13 with neomycin. The analysis shows that compound 13 has potentiating effect with neomycin in inhibiting the growth of S. aureus Mu3 strain. Heat Plot shows the average growth from replicated experiments.

FIG. 5 shows a microdilution checkerboard analysis showing the combined effect of compound 13 with spectinomycin. The analysis shows that compound 13 has potentiating effect with spectinomycin in inhibiting the growth of S. aureus Mu3 strain. Heat Plot shows the average growth from replicated experiments.

FIG. 6 shows a microdilution checkerboard analysis showing the combined effect of compound 13 with neamine. The analysis shows that compound 13 has potentiating effect with neamine in inhibiting the growth of S. aureus Mu3 strain. Heat Plot shows the average growth from replicated experiments.

FIG. 7 shows a microdilution checkerboard analysis showing the combined effect of compound 13 with gentamicin. The analysis shows that compound 13 has potentiating effect with gentamicin in inhibiting the growth of S. aureus ATCC 29213 strain. Heat Plot shows the average growth from replicated experiments.

FIG. 8 shows a microdilution checkerboard analysis showing the combined effect of compound 13 with gentamicin. The heat map plot shows normalized growth of checkerboard assay of compound 13 with Gentamicin against Newman. The analysis shows that compound 13 has potentiating effect with gentamicin in inhibiting the growth of S. aureus Newman strain. Heat Plot shows the average growth from replicated experiments.

FIG. 9 shows a microdilution checkerboard analysis showing the combined effect of compound 13 with gentamicin. The heat map plot shows normalized growth of checkerboard assay of compound 13 with Gentamicin against RN4220. The analysis shows that compound 13 has potentiating effect with gentamicin in inhibiting the growth of S. aureus RN4220 strain. Heat Plot shows the average growth from replicated experiments.

FIG. 10 shows a microdilution checkerboard analysis showing the combined effect of compound 13 with gentamicin. The heat map plot shows normalized growth of checkerboard assay of compound 13 with Gentamicin against USA300.The analysis shows that compound 13 has potentiating effect with gentamicin in inhibiting the growth of S. aureus USA300 strain. Heat Plot shows the average growth from replicated experiments.

FIG. 11 shows a microdilution checkerboard analysis showing the combined effect of compound 13 with gentamicin. The heat map plot shows normalized growth of checkerboard assay of compound 13 with Gentamicin against COL. The analysis shows that compound 13 has potentiating effect with gentamicin in inhibiting the growth of S. aureus COL strain. Heat Plot shows the average growth from replicated experiments.

FIG. 12 shows a microdilution checkerboard analysis showing the combined effect of compound 13 with gentamicin. The heat map plot shows normalized growth of checkerboard assay of compound 13 with Gentamicin against RN6390.The analysis shows that compound 13 has potentiating effect with gentamicin in inhibiting the growth of S. aureus RN6390 strain. Heat Plot shows the average growth from replicated experiments.

FIG. 13 shows a microdilution checkerboard analysis showing the combined effect of compound 13 with gentamicin. The analysis shows that compound 13 has potentiating effect with gentamicin in inhibiting the growth of S. aureus ST45-502 strain. Heat Plot shows the average growth from replicated experiments.

FIG. 14 shows a microdilution checkerboard analysis showing the combined effect of compound 13 with gentamicin. The analysis shows that compound 13 has potentiating effect with gentamicin in inhibiting the growth of S. aureus AE-052 strain. Heat Plot shows the average growth from replicated experiments.

FIG. 15 shows a microdilution checkerboard analysis showing the combined effect of compound 13 with gentamicin. The analysis shows that compound 13 has potentiating effect with gentamicin in inhibiting the growth of S. aureus ST239-III-AH-504 strain. Heat Plot shows the average growth from replicated experiments.

FIG. 16 shows a microdilution checkerboard analysis showing the combined effect of compound 13 with gentamicin. The analysis shows that compound 13 has potentiating effect with gentamicin in inhibiting the growth of S. aureus ST239-IIIA-503 strain. Heat Plot shows the average growth from replicated experiments.

FIGS. 17A-17D shows secondary screenings of compound 13 analogues at 50 μM in combination of various concentrations of gentamicin against S. aureus Mu3. The analysis shows that some structural analogues possess the combinatory inhibitory effect against S. aureus Mu3. Heat Plots show the average growth from replicated experiments.

FIGS. 18A-18D shows secondary screenings of compound 13 analogues at 20 μM in combination of various concentrations of gentamicin against S. aureus Mu3. The analysis shows that some structural analogues possess the combinatory inhibitory effect against S. aureus Mu3. Heat Plots show the average growth from replicated experiments.

FIGS. 19A-19D shows secondary screenings of compound 13 analogues at 5 μM in combination of various concentrations of gentamicin against S. aureus Mu3. The analysis shows that some structural analogues possess the combinatory inhibitory effect against S. aureus Mu3. Heat Plots show the average growth from replicated experiments.

FIG. 20 shows a microdilution checkerboard analysis showing the combined effect of compound 13 with gentamicin. The analysis shows that compound 13 has potentiating effect with gentamicin in inhibiting the growth of S. aureus Mu3 strain. Heat Plot shows the average growth from replicated experiments.

FIG. 21 shows a microdilution checkerboard analysis showing the combined effect of compound 17 with gentamicin. The analysis shows that compound 17 has potentiating effect with gentamicin in inhibiting the growth of S. aureus Mu3 strain. Heat Plot shows the average growth from replicated experiments.

FIG. 22 shows a microdilution checkerboard analysis showing the combined effect of compound 19 with gentamicin. The analysis shows that compound 19 has potentiating effect with gentamicin in inhibiting the growth of S. aureus Mu3 strain. Heat Plot shows the average growth from replicated experiments.

FIG. 23 shows a microdilution checkerboard analysis showing the combined effect of compound 20 with gentamicin. The analysis shows that compound 20 has potentiating effect with gentamicin in inhibiting the growth of S. aureus Mu3 strain. Heat Plot shows the average growth from replicated experiments.

FIG. 24 shows a microdilution checkerboard analysis showing the combined effect of compound 36 with gentamicin. The analysis shows that compound 36 has potentiating effect with gentamicin in inhibiting the growth of S. aureus Mu3 strain. Heat Plot shows the average growth from replicated experiments.

FIG. 25 shows a microdilution checkerboard analysis showing the combined effect of compound 82 with gentamicin. The analysis shows that compound 82 has potentiating effect with gentamicin in inhibiting the growth of S. aureus Mu3 strain. Heat Plot shows the average growth from replicated experiments.

FIG. 26 Treatment of compound 3 reduce kidney bacterial load in BALB/c mice after Mu3 infection. The analysis shows that compound 3 can reduce bacterial infection by itself.

FIG. 27 Treatment of compound 3 reduce spleen bacterial load in BALB/c mice after Mu3 infection. The analysis shows that compound 3 can reduce bacterial infection by itself.

FIG. 28 Treatment of compound 3 reduce liver bacterial load in BALB/c mice after Mu3 infection. The analysis shows that compound 3 can reduce bacterial infection by itself.

FIG. 29 Treatment of compound 3 reduce spleen bacterial load in BALB/c mice after USA300 infection. The analysis shows that compound 3 can reduce bacterial infection by itself.

FIG. 30 Treatment of compound 3 reduce liver bacterial load in BALB/c mice after USA300 infection. The analysis shows that compound 3 can reduce bacterial infection by itself.

FIG. 31 Heat map plot showing normalized growth of checkerboard assay of compound 82 with Gentamicin against L. monocytogenes. The analysis shows that compound 82 has potentiating effect with gentamicin in inhibiting the growth of L. monocytogenes strain. Heat Plot shows the average growth from replicated experiments.

FIG. 32 Heat map plot showing normalized growth of checkerboard assay of compound 82 with Gentamicin against E. faecalis. The analysis shows that compound 82 has potentiating effect with gentamicin in inhibiting the growth of E. faecalis strain. Heat Plot shows the average growth from replicated experiments.

FIG. 33 Heat map plot showing normalized growth of checkerboard assay of compound 82 with Gentamicin against E. coli. The analysis shows that compound 82 has potentiating effect with gentamicin in inhibiting the growth of E. coli strain. Heat Plot shows the average growth from replicated experiments.

FIG. 34 Heat map plot showing normalized growth of checkerboard assay of compound 82 with Gentamicin against S. pneumoniae. The analysis shows that compound 82 has potentiating effect with gentamicin in inhibiting the growth of S. pneumonia strain. Heat Plot shows the average growth from replicated experiments.

FIG. 35 shows a microdilution checkerboard analysis showing the combined effect of compound 82 with neamine. The analysis shows that compound 82 has potentiating effect with neamine in inhibiting the growth of S. aureus Mu3 strain. Heat Plot shows the average growth from replicated experiments.

### DETAILED DESCRIPTION OF THE INVENTION

### I. Definitions

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

“Aerosol” as used herein refers to any preparation of a fine mist of particles, which can be in solution or a suspension, whether or not it is produced using a propellant.

The term “alkyl” refers to the radical of saturated aliphatic groups (i.e., an alkane with one hydrogen atom removed), including straight-chain alkyl groups, branched-chain alkyl groups, cycloalkyl (alicyclic) groups, alkyl-substituted cycloalkyl groups, and cycloalkyl-substituted alkyl groups.

“Amphiphilic” as used herein refers to a molecule combining hydrophilic and lipophilic (hydrophobic) properties.

The term “carrier” refers to a diluent, adjuvant, excipient, and/or vehicle with which the compound and/or antibiotic are administered.

“Cleaning formulation”, as used herein, means a composition suitable for application to a surface for removing dirt, oils, and the like, for disinfecting, or a combination thereof. Cleaning compositions can be antibacterial, antimicrobial, or both. Cleaning compositions can be suitable for use on the human skin meaning that all components of the composition are present at concentrations that cause no significant signs of irritation when applied to human skin. As used herein, “significant signs of irritation” include erythema, redness, and/or swelling at the site of injection at the site of application, necrosis at the site of application, exfoliative dermatitis at the site of application, and severe pain that prevents daily activity and/or requires medical attention or hospitalization.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

A “cream” is a viscous liquid or semi-solid emulsion of either the “oil-in-water” or “water-in-oil type”.

An “emulsion” is a composition containing a mixture of non-miscible components homogenously blended together.

“Gel” as used herein is a colloid in which the dispersed phase has combined with the continuous phase to produce a semisolid material, such as jelly.

“Hydrophilic” as used herein refers to substances that have strongly polar groups that readily interact with water.

“Hydrophobic” as used herein refers to substances that lack an affinity for water; tending to repel and not absorb water as well as not dissolve in or mix with water.

“Lipophilic” as used herein refers to compounds having an affinity for lipids.

A “lotion” is a low-to medium-viscosity liquid formulation.

“Oil” as used herein refers to a composition containing at least 95% wt. of a lipophilic substance. Examples of lipophilic substances include but are not limited to naturally occurring and synthetic oils, fats, fatty acids, lecithins, triglycerides and combinations thereof.

An “ointment” is a semisolid preparation containing an ointment base and optionally one or more active agents.

“Parenteral administration”, as used herein, means administration by any method other than through the digestive tract or non-invasive topical or regional routes.

The term “pharmaceutically acceptable”, as used herein with regard to pharmaceutical compositions, means approved by a regulatory agency of the Federal or a state government or listed in the U.S. Pharmacopeia or other generally recognized pharmacopeia for use in animals and/or in humans.

“Pharmaceutically acceptable salt”, as used herein, refers to derivatives of the compounds defined herein, wherein the parent compound is modified by making acid or base salts thereof.

As used herein, “treatment” or “treating” refers to arresting or inhibiting, or attempting to arrest or inhibit, the development or progression of an infection and/or causing, or attempting to cause, the reduction, suppression, regression, or remission of an infection and/or a symptom thereof. As would be understood by those skilled in the art, various clinical and scientific methodologies and assays may be used to assess the development or progression of an infection, and similarly, various clinical and scientific methodologies and assays may be used to assess the reduction, regression, or remission of an infection or its symptoms. “Treatment” refers to both therapeutic treatment and prophylactic or preventative measures. Those in need of treatment include those already with the infection as well as those prone to have the infection or those in whom the infection is to be prevented. In at least some forms, the infection being treated is Staphylococcus aureus, Pseudomonas aeruginosa, Listeria monocytogenes, Burkholderia cepacia, Escherichia coli, Enterococcus faecalis, Streptococcus pneumoniae infection. In other forms, the infection being treated is a microbial infection.

As used herein, the term “subject” refers to an animal. Typically, the terms “subject” and “patient” may be used interchangeably herein in reference to a subject. As such, a “subject” includes a human that is being treated for a microbial infection as a patient.

The term “animal,” includes, but is not limited to, mouse, rat, dog, cat, rabbit, pig, monkey, chimpanzee, and human.

The terms “effective amount” and “therapeutically effective amount,” used interchangeably, as applied to the compounds, antibiotics, and pharmaceutical compositions described herein, mean the quantity necessary to render the desired therapeutic result. For example, an effective amount is a level effective to treat, cure, or alleviate the symptoms of an infection for which the composition and/or antibiotic, or pharmaceutical composition, is/are being administered. Amounts effective for the particular therapeutic goal sought will depend upon a variety of factors including the infection being treated and its severity and/or stage of development/progression; the bioavailability and activity of the specific compound and/or antibiotic, or pharmaceutical composition, used; the route or method of administration and introduction site on the subject; the rate of clearance of the specific composition and other pharmacokinetic properties; the duration of treatment; inoculation regimen; drugs used in combination or coincident with the specific composition; the age, body weight, sex, diet, physiology and general health of the subject being treated; and like factors well known to one of skill in the relevant scientific art. Some variation in dosage will necessarily occur depending upon the condition of the subject being treated, and the physician or other individual administering treatment will, in any event, determine the appropriate dosage for an individual patient.

### II. Compositions

High throughput screening provides a good opportunity for discovery of new small molecules as antibacterial agents.

Through sequential screening processes, a chemical compound library to a lead compound for future chemical optimization for antibiotics potentiators was identified. As shown below, compound 13 demonstrated the potentiating effect in combination with aminoglycoside, gentamicin, kanamycin, amikacin, neomycin, spectinomycin and neamine, which are commonly used in clinical settings. Furthermore, these compounds (at a concentration of around 50 μM) were shown to reduce the MIC of the antibiotic below the corresponding clinical breakpoint, thus making an ineffective antibiotic usable again for MRSA treatment. In some forms, the compound potentiates the therapeutic effects of one or more antimicrobial agents by at least 2 fold when they are co-administered. Potentiation can be determined in vitro, as described in the Examples, by measuring bacterial growth inhibition in the presence of the disclosed compounds when administered with the antibiotic. In some forms, the compound is effective to inhibit bacterial growth by at least 30%, preferably at least 40%, and more preferably at least 60% more than the inhibition obtained with gentamycin alone (gentamycin+DMSO control). In the other forms the compounds have antivirulence properties, measured as repression in hla promoter lux reporter assay when compared to gentamycin+DMSO control. Suppressions of pigment production were also observed in some pigment producing bacteria in the presence of the compounds. In some aspects, the compounds effective to inhibit dihydrofolate reductase in a microorganism.

It is within the abilities for those of ordinary skill in the art to use the data obtained from cell culture assays and animal studies to formulate a dosage range for use in humans. The dosage of such compounds lies preferably within a range of circulating concentrations that include the ED₅₀ with little or no toxicity. The dosage can vary within his range depending upon the dosage form employed and the route of administration. For any compound used in the disclosed methods, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a modulator is from about 1 ng/kg to 10 mg/kg for a typical subject.

Accordingly, compounds with antimicrobial activity are disclosed which can be used in an effective amount in a composition to prevent, treat or ameliorate the symptoms resulting from a microbial infection. In some forms, the compounds potentiate the antimicrobial effects of antibiotics. In another form, the compounds possess antivirulence properties.

The compounds disclosed herein can be utilized in combination with other bioactive agents, for example, antibiotics during treatment. Such combination treatments may further enhance the therapeutic effects of treatment with the disclosed compound(s). The disclosed compounds can provide additive, synergistic or antibiotic potentiating effects with antibiotics.

A. Compounds

The disclosed compounds are represented by the following generic Formula I:

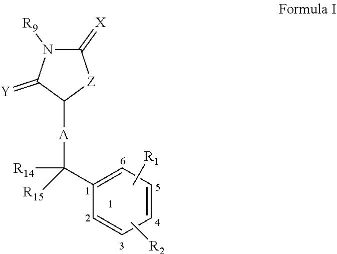

where X and Y are independently O, S, or NR₁₀;

where Z is O, S, CR₁₁R₁₂, or NR₁₃;

where R₁, R_{2,} R_{9,} R_{10,} R_{11,} R_{12,} R_{13,} R_{14,} and R_{15,} are independently absent, hydrogen, —C(O)R₁₈, —C(W)NR₁₉R_{20,} or substituted or unsubstituted alkyl, alkenyl, alkynyl, alkoxy, cycloalkyl, heterocyclyl, cycloalkenyl, heterocycloalkenyl, aryl, heteroaryl, aroxy, arylalkyl, heteroalkyl, alkylaryl, halogen, alkylheteroaryl, —N₁₆R₁₇; wherein R₁₆ and R₁₇ are independently hydrogen, substituted or unsubstituted alkyl, alkenyl, alkynyl, cycloalkyl, heterocyclyl, cycloalkenyl, heterocycloalkenyl, aryl, heteroaryl, arylalkyl, heteroalkyl, alkylaryl, or alkylheteroaryl;

where R₁₈ is hydrogen, hydroxyl, or substituted or unsubstituted alkyl, alkenyl, alkynyl, cycloalkyl, alkoxy, heterocyclyl, cycloalkenyl, heterocycloalkenyl, aryl, aroxy, heteroaryl, arylalkyl, heteroalkyl, alkylaryl, or alkylheteroaryl;

where W is O, S, or NR_{21;}

where R_{19,} R_{20,} and R₂₁ are independently hydrogen or substituted or unsubstituted alkyl, alkenyl, alkynyl, cycloalkyl, heterocyclyl, cycloalkenyl, heterocycloalkenyl, aryl, heteroaryl, arylalkyl, heteroalkyl, alkylaryl, or alkylheteroaryl;

where A is a double bond or single bond; and where R₁₅ is absent when A is a double bond.

In some forms, the compound can be

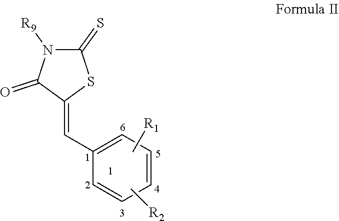

where R₁ is —O—R_{4,} —NR₅R_{8,} or —CH₂—R_{5;} R₂ is halogen, absent, —O—CH_{3,} or —O—CH₂—CH_{3;} R₉ is —COOH, —CH₂—COOH, —CH₂—O—CH_{3,} or —CH₂—CH₂—O—CH_{3;} R₄ is —CH₂—R₅ or —CO—R_{5;} R₅ is

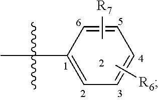

R₆ is a halogen or absent; and R₇ is halogen or absent.

In some forms of the compound, R₁ is at a position selected from the group consisting of 2, 3, 4, 5, and 6. In some forms, R₆ is at position 4. In some forms, R₇ is at position 2 or 6. In some forms, R₂ is at position 3, 4, or 5. In some forms, R₆ is at position 4, R₇ is at position 2 or 6, and R₂ is at position 3 or 5.

In some forms, the compound is

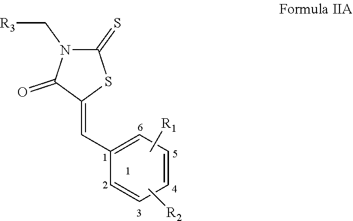

where R₁ is —O—R_{4,} —NR₅R_{8,} or —CH₂—R_{5;} R₂ is halogen, absent, —O—CH_{3,} or —O—CH₂—CH_{3;} R₃ is —COOH or —CH₂—COOH; R₄ is —CH₂—R₅ or —CO—R_{5;} R₅ is

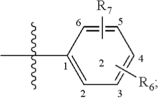

R₆ is halogen or absent; and R₇ is halogen or absent.

In some forms, R₁ is at a position selected from the group consisting of 2, 3, 4, 5, and 6. In some forms, R₆ is at position 4. In some forms, R₇ is at position 2 or 6. In some forms, R₂ is at position 3, 4, or 5. In some forms, R₆ is at position 4, R₇ is at position 2 or 6, and R₂ is at position 3 or 5.

In some forms, R₁ is —O—R₄ and is at position 3; R₂ is not present; R₃ is —COOH; R₄ is —CH₂—R_{5;} R₅ is

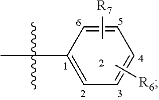

R₆ is —Cl and is at position 4; and R₇ is —Cl and is at position 2.

In some forms, R₁ is —O—R₄ and is at position 3; R₂ is not present; R₃ is —COOH; R₄ is —CH₂—R_{5;} R₅ is

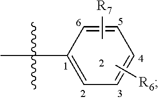

R₆ is —Cl; and R₇ is absent.

In some forms, R₁ is —O—R_{4,} and is at position 4; R₂ is not present; R₃ is —COOH; R₄ is —CH₂—R_{5;} R₅ is

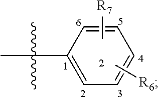

R₆ is —Cl; and R₇ is absent. In some forms, R₁ is —O—R_{4,} and is at position 4; R₂ is —O—CH₂—CH_{3;} R₃ is COOH;

R₄ is —CH₂—R_{5;} R₅ is

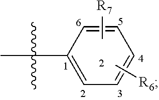

R₆ is—Cl; and R₇ is absent.

These and other groups are listed in Table 8

TABLE 8
Examples of Substituents for Formula IIA

| | A | B | C | D | E | F | G |
|---|---|---|---|---|---|---|---|
| R₁ | —O—R₄ | —O—R₄ | —O—R₄ | —O—R₄ | —O—R₄ | —O—R₄ | —O—R₄ |
| R₁ ring | 4 | 4 | 4 | 4 | 4 | 4 | 4 |
| position | | | | | | | |
| R₂ | — | — | — | — | — | — | — |
| R₂ ring | — | — | — | — | — | — | — |
| position | | | | | | | |
| R₃ | —COOH | —COOH | —COOH | —COOH | —COOH | —COOH | —COOH |
| R₄ | —CH₂—R₅ | —CH₂—R₅ | —CH₂—R₅ | —CH₂—R₅ | —CH₂—R₅ | —CH₂—R₅ | —CH₂—R₅ |
| R₅ | benzyl | benzyl | benzyl | benzyl | benzyl | benzyl | benzyl |
| R₆ | —Cl | —Cl | —Cl | —Cl | —Cl | —Cl | —I |
| R₆ ring | 4 | 4 | 4 | 2 | 2 | 3 | 4 |
| position | | | | | | | |
| R₇ | — | —Cl | —Cl | — | —Cl | — | — |
| R₇ ring | — | 2 | 3 | — | 3 | — | — |
| position | | | | | | | |
| | H | I | J | K | L | M | N |
| R₁ | —O—R₄ | —O—R₄ | —O—R₄ | —O—R₄ | —O—R₄ | —O—R₄ | —O—R₄ |
| R₁ ring | 4 | 3 | 3 | 3 | ³ | 3 | 3 |
| position | | | | | | | |
| R₂ | — | — | — | — | — | — | — |
| R₂ ring | — | — | — | — | — | — | — |
| position | | | | | | | |
| R₃ | —COOH | —COOH | —COOH | —COOH | —COOH | —COOH | —COOH |
| R₄ | —CH₂—R₅ | —CH₂—R₅ | —CH₂—R₅ | —CH₂—R₅ | —CH₂—R₅ | —CH₂—R₅ | —CH₂—R₅ |
| R₅ | benzyl | benzyl | benzyl | benzyl | benzyl | benzyl | benzyl |
| R₆ | —CH₃ | —Cl | —Cl | —Cl | —Cl | —Cl | —Cl |
| R₆ ring | 4 | 4 | 4 | 4 | 2 | 2 | 3 |
| position | | | | | | | |
| R₇ | — | — | —Cl | —Cl | — | —Cl | — |
| R₇ ring | — | — | 2 | 3 | — | 3 | — |
| position | | | | | | | |
| | O | P | Q | R | S | T | U |
| R₁ | —O—R₄ | —O—R₄ | —O—R₄ | —O—R₄ | —O—R₄ | —O—R₄ | —O—R₄ |
| R₁ ring | 3 | 3 | 2 | 2 | 2 | 2 | 2 |
| position | | | | | | | |
| R₂ | — | — | — | — | — | — | — |
| R₂ ring | — | — | — | — | — | — | — |
| position | | | | | | | |
| R₃ | —COOH | —COOH | —COOH | —COOH | —COOH | —COOH | —COOH |
| R₄ | —CH₂—R₅ | —CH₂—R₅ | —CH₂—R₅ | —CH₂—R₅ | —CH₂—R₅ | —CH₂—R₅ | —CH₂—R₅ |
| R₅ | benzyl | benzyl | benzyl | benzyl | benzyl | benzyl | benzyl |
| R₆ | —I | —CH₃ | —Cl | —Cl | —Cl | —Cl | —Cl |
| R₆ ring | 4 | 4 | 4 | 4 | 4 | 2 | 2 |
| position | | | | | | | |
| R₇ | — | — | — | —Cl | —Cl | — | —Cl |
| R₇ ring | — | — | — | 2 | 3 | — | 3 |
| position | | | | | | | |
| | V | W | X | Y | Z | AA | AB |
| R₁ | —O—R₄ | —O—R₄ | —O—R₄ | —O—R₄ | —O—R₄ | —O—R₄ | —O—R₄ |
| R₁ ring | 2 | 2 | 2 | 4 | 4 | 4 | 4 |
| position | | | | | | | |
| R₂ | — | — | — | —O—CH₃ | —O—CH₃ | —O—CH₃ | —O—CH₃ |
| R₂ ring | — | — | — | 3 | 3 | 3 | 3 |
| position | | | | | | | |
| R₃ | —COOH | —COOH | —COOH | —COOH | —COOH | —COOH | —COOH |
| R₄ | —CH₂—R₅ | —CH₂—R₅ | —CH₂—R₅ | —CH₂—R₅ | —CH₂—R₅ | —CH₂—R₅ | —CH₂—R₅ |
| R₅ | benzyl | benzyl | benzyl | benzyl | benzyl | benzyl | benzyl |
| R₆ | —Cl | —I | —CH₃ | —Cl | —Cl | —Cl | —Cl |
| R₆ ring | 3 | 4 | 4 | 4 | 4 | 4 | 2 |
| position | | | | | | | |
| R₇ | — | — | — | — | —Cl | —Cl | — |
| R₇ ring | — | — | — | — | 2 | 3 | — |
| position | | | | | | | |

| | AC | AD | AE | AF | AG | AH | AI |
|---|---|---|---|---|---|---|---|
| R₁ | —O—R₄ | —O—R₄ | —O—R₄ | —O—R₄ | —O—R₄ | —O—R₄ | —O—R₄ |
| R₁ ring | 4 | 4 | 4 | 4 | 4 | 4 | 4 |
| position | | | | | | | |
| R₂ | —O—CH₃ | —O—CH₃ | —O—CH₃ | —O—CH₃ | —OCH₂—CH₃ | —OCH₂—CH₃ | —OCH₂—CH₃ |
| R₂ ring | 3 | 3 | 3 | 3 | 3 | 3 | 3 |
| position | | | | | | | |
| R₃ | —COOH | —COOH | —COOH | —COOH | —COOH | —COOH | —COOH |
| R₄ | —CH₂—R₅ | —CH₂—R₅ | —CH₂—R₅ | —CH₂—R₅ | —CH₂—R₅ | —CH₂—R₅ | —CH₂—R₅ |
| R₅ | benzyl | benzyl | benzyl | benzyl | benzyl | benzyl | benzyl |
| R₆ | —Cl | —Cl | —I | —CH₃ | —Cl | —Cl | —Cl |
| R₆ ring | 2 | 3 | 4 | 4 | 4 | 4 | 4 |
| position | | | | | | | |
| R₇ | —Cl | — | — | — | — | —Cl | —Cl |
| R₇ ring | 3 | — | — | — | — | 2 | 3 |
| position | | | | | | | |

| | AJ | AK | AL | AM | AN |
|---|---|---|---|---|---|
| R₁ | —O—R₄ | —O—R₄ | —O—R₄ | —O—R₄ | —O—R₄ |
| R₁ ring | 4 | 4 | 4 | 4 | 4 |
| position | | | | | |
| R₂ | —O—CH₂—CH₃ | —O—CH₂—CH₃ | —O—CH₂—CH₃ | —O—CH₂—CH₃ | —O—CH₂—CH₃ |
| R₂ ring | 3 | 3 | 3 | 3 | 3 |
| position | | | | | |
| R₃ | —COOH | —COOH | —COOH | —COOH | —COOH |
| R₄ | —CH₂—R₅ | —CH₂—R₅ | —CH₂—R₅ | —CH₂—R₅ | —CH₂—R₅ |
| R₅ | benzyl | benzyl | benzyl | benzyl | benzyl |
| R₆ | —Cl | —Cl | —Cl | —I | —CH₃ |
| R₆ ring | 2 | 2 | 3 | 4 | 4 |
| position | | | | | |
| R₇ | — | —Cl | — | — | — |
| R₇ ring | — | 3 | — | — | — |
| position | | | | | |

| | BA | BB | BC | BD | BE | BF | BG |
|---|---|---|---|---|---|---|---|
| R₁ | —O—R₄ | —O—R₄ | —O—R₄ | —O—R₄ | —O—R₄ | —O—R₄ | —O—R₄ |
| R₁ ring | 4 | 4 | 4 | 4 | 4 | 4 | 4 |
| position | | | | | | | |
| R₂ | — | — | — | — | — | — | — |
| R₂ ring | — | — | — | — | — | — | — |
| position | | | | | | | |
| R₃ | —COOH | —COOH | —COOH | —COOH | —COOH | —COOH | —COOH |
| R₄ | —CO—R₅ | —CO—R₅ | —CO—R₅ | —CO—R₅ | —CO—R₅ | —CO—R₅ | —CO—R₅ |
| R₅ | benzyl | benzyl | benzyl | benzyl | benzyl | benzyl | benzyl |
| R₆ | —Cl | —Cl | —Cl | —Cl | —Cl | —Cl | —Cl |
| R₆ ring | 4 | 4 | 4 | 2 | 2 | 3 | 4 |
| position | | | | | | | |
| R₇ | — | —Cl | —Cl | — | —Cl | — | — |
| R₇ ring | — | 2 | 3 | — | 3 | — | — |
| position | | | | | | | |
| | BH | BI | BJ | BK | BL | BM | BN |
| R₁ | —O—R₄ | —O—R₄ | —O—R₄ | —O—R₄ | —O—R₄ | —O—R₄ | —O—R₄ |
| R₁ ring | 4 | 3 | 3 | 3 | 3 | 3 | 3 |
| position | | | | | | | |
| R₂ | — | — | — | — | — | — | — |
| R₂ ring | — | — | — | — | — | — | — |
| position | | | | | | | |
| R₃ | —COOH | —COOH | —COOH | —COOH | —COOH | —COOH | —COOH |
| R₄ | —CO—R₅ | —CO—R₅ | —CO—R₅ | —CO—R₅ | —CO—R₅ | —CO—R₅ | —CO—R₅ |
| R₅ | benzyl | benzyl | benzyl | benzyl | benzyl | benzyl | benzyl |
| R₆ | —CH₃ | —Cl | —Cl | —Cl | —Cl | —Cl | —Cl |
| R₆ ring | 4 | 4 | 4 | 4 | 2 | 2 | 3 |
| position | | | | | | | |
| R₇ | — | — | —Cl | —Cl | — | —Cl | — |
| R₇ ring | — | — | 2 | 3 | — | 3 | — |
| position | | | | | | | |
| | BO | BP | BQ | BR | BS | BT | BU |
| R₁ | —O—R₄ | —O—R₄ | —O—R₄ | —O—R₄ | —O—R₄ | —O—R₄ | —O—R₄ |
| R₁ ring | 3 | 3 | 2 | 2 | 2 | 2 | 2 |
| position | | | | | | | |
| R₂ | — | — | — | — | — | — | — |
| R₂ ring | — | — | — | — | — | — | — |
| position | | | | | | | |
| R₃ | —COOH | —COOH | —COOH | —COOH | —COOH | —COOH | —COOH |
| R₄ | —CO—R₅ | —CO—R₅ | —CO—R₅ | —CO—R₅ | —CO—R₅ | —CO—R₅ | —CO—R₅ |
| R₅ | benzyl | benzyl | benzyl | benzyl | benzyl | benzyl | benzyl |
| R₆ | —I | —CH₃ | —Cl | —Cl | —Cl | —Cl | —Cl |
| R₆ ring | 4 | 4 | 4 | 4 | 4 | 2 | 2 |
| position | | | | | | | |
| R₇ | — | — | — | —Cl | —Cl | — | —Cl |
| R₇ ring | — | — | — | 2 | 3 | — | 3 |
| position | | | | | | | |
| | BV | BW | BX | BY | BZ | CA | CB |
| R₁ | —O—R₄ | —O—R₄ | —O—R₄ | —O—R₄ | —O—R₄ | —O—R₄ | —O—R₄ |
| R₁ ring | 2 | 2 | 2 | 4 | 4 | 4 | 4 |
| position | | | | | | | |
| R₂ | — | — | — | —O—CH₃ | —O—CH₃ | —O—CH₃ | —O—CH₃ |
| R₂ ring | — | — | — | 3 | 3 | 3 | 3 |
| position | | | | | | | |
| R₃ | —COOH | —COOH | —COOH | —COOH | —COOH | —COOH | —COOH |
| R₄ | —CO—R₅ | —CO—R₅ | —CO—R₅ | —CO—R₅ | —CO—R₅ | —CO—R₅ | —CO—R₅ |
| R₅ | benzyl | benzyl | benzyl | benzyl | benzyl | benzyl | benzyl |
| R₆ | —Cl | —I | —CH₃ | —Cl | —Cl | —Cl | —Cl |
| R₆ ring | 3 | 4 | 4 | 4 | 4 | 4 | 2 |
| position | | | | | | | |
| R₇ | — | — | — | — | —Cl | —Cl | — |
| R₇ ring | — | — | — | — | 2 | 3 | — |
| position | | | | | | | |

| | CD | CE | CF | CG | CH | CI | CJ |
|---|---|---|---|---|---|---|---|
| R₁ | —O—R₄ | —O—R₄ | —O—R₄ | —O—R₄ | —O—R₄ | —O—R₄ | —O—R₄ |
| R₁ ring | 4 | 4 | 4 | 4 | 4 | 4 | 4 |
| position | | | | | | | |
| R₂ | —O—CH₃ | —O—CH₃ | —O—CH₃ | —O—CH₃ | —O—CH₂—CH₃ | —O—CH₂—CH₃ | —O—CH₂—CH₃ |
| R₂ ring | 3 | 3 | 3 | 3 | 3 | 3 | 3 |
| position | | | | | | | |
| R₃ | —COOH | —COOH | —COOH | —COOH | —COOH | —COOH | —COOH |
| R₄ | —CO—R₅ | —CO—R₅ | —CO—R₅ | —CO—R₅ | —CO—R₅ | —CO—R₅ | —CO—R₅ |
| R₅ | benzyl | benzyl | benzyl | benzyl | benzyl | benzyl | benzyl |
| R₆ | —Cl | —Cl | —I | —CH₃ | —Cl | —Cl | —Cl |
| R₆ ring | 2 | 3 | 4 | 4 | 4 | 4 | 4 |
| position | | | | | | | |
| R₇ | —Cl | — | — | — | — | —Cl | —Cl |
| R₇ ring | 3 | — | — | — | — | 2 | 3 |
| position | | | | | | | |

| | CK | CL | CM | CN | CO |
|---|---|---|---|---|---|
| R₁ | —O—R₄ | —O—R₄ | —O—R₄ | —O—R₄ | —O—R₄ |
| R₁ ring | 4 | | 4 | 4 | 4 |
| position | | | | | |
| R₂ | —O—CH₂—CH₃ | —O—CH₂—CH₃ | —O—CH₂—CH₃ | —O—CH₂—CH₃ | —O—CH₂—CH₃ |
| R₂ ring | 3 | 3 | 3 | 3 | 3 |
| position | | | | | |
| R₃ | —COOH | —COOH | —COOH | —COOH | —COOH |
| R₄ | —CO—R₅ | —CO—R₅ | —CO—R₅ | —CO—R₅ | —CO—R₅ |
| R₅ | benzyl | benzyl | benzyl | benzyl | benzyl |
| R₆ | —Cl | —Cl | —Cl | —I | —CH₃ |
| R₆ ring | 2 | 2 | 3 | 4 | 4 |
| position | | | | | |
| R₇ | — | —Cl | — | — | — |
| R₇ ring | — | 3 | — | — | — |
| position | | | | | |

All combinations of R₁-R₇ disclosed above are contemplated.

In some forms, X and Y are independently O, S, or NR_{10;} Z is O, S, CR₁₁R_{12,} or N_{13;} R₁, R_{2,} R_{9,} R_{10,} R₁₁, R_{12,} R_{13,} R_{14,} and R_{15,} are independently absent, hydrogen, substituted or unsubstituted alkyl, alkenyl, alkynyl, alkoxy, cycloalkyl, heterocyclyl, cycloalkenyl, heterocycloalkenyl, aryl, heteroaryl, aroxy, arylalkyl, heteroalkyl, alkylaryl, halogen, alkylheteroaryl, —N₁₆R₁₇; wherein R₁₆ and R₁₇ are independently hydrogen, substituted or unsubstituted alkyl, alkenyl, alkynyl, cycloalkyl, heterocyclyl, cycloalkenyl, heterocycloalkenyl, aryl, heteroaryl, arylalkyl, heteroalkyl, alkylaryl, alkylheteroaryl, —C(O)R₁₈, or —C(W)NR₁₉R_{20;} R₁₈ is hydrogen, hydroxyl, substituted or unsubstituted alkyl, alkenyl, alkynyl, cycloalkyl, alkoxy, heterocyclyl, cycloalkenyl, heterocycloalkenyl, aryl, aroxy, heteroaryl, arylalkyl, heteroalkyl, alkylaryl, or alkylheteroaryl; W is O, S, NR_{21;} R_{19,} R_{20,} and R₂₁ are independently hydrogen, substituted or unsubstituted alkyl, alkenyl, alkynyl, cycloalkyl, heterocyclyl, cycloalkenyl, heterocycloalkenyl, aryl, heteroaryl, arylalkyl, heteroalkyl, alkylaryl, or alkylheteroaryl; A is a double bond or single bond; and when A is a double bond, R₁₅ is absent.

In some forms of Formula II, R₁ can be —O—R₄ or —NR₅R₈ or —CH₂—R_{5;} R₂ can be halogen or is absent or —O—CH₃ or —O—CH₂—CH₃; R₉ can be —COOH or —CH₂—COOH or —CH₂—O—CH₃ or —CH₂—CH₂—O—CH_{3;} R₄ can be —CH₂—R₅ or —CO—R_{5;} R₅ can be

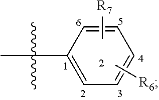

R₆ can be a halogen or is absent; R₇ can be halogen or is absent. In some forms, R₁ is at position 3 or 5; R₁ is at position 4; R₁ is at position 2 or 6; in some forms, R₆ is at position 4. In some forms, R₇ is at position 2 or 6. In some forms, R₂ is at position 3, 4, or 5.

All combinations of R₁-R₇ disclosed above are contemplated.

In some forms the compounds are represented by the Formula IIA:

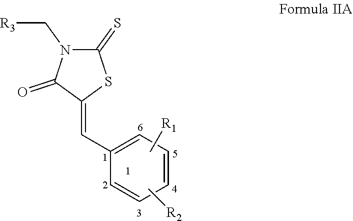

In these forms, R₁ can be —O—R₄ or —NR₅R₈ or —CH₂—R_{5;} R₂ can be a halogen, is absent or it can be —O—CH₃ or —O—CH₂—CH_{3;} R₃ can be —COOH or —CH₂—COOH; R₄ can be —CH₂—R₅ or —CO—R_{5;} R₅ can be

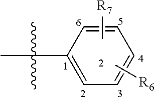

In some forms, R₆ is halogen or absent. In some forms, R₇ is halogen or absent. In some forms, R₁ is at position 3 or 5. In some forms, R₁ is at position 4. In some forms, R₁ is at position 2 or 6. In some forms, R₆ is at position 4. In some forms, R₇ is at position 2 or 6. In some forms, R₂ is at position 3, 4, or 5.

All combinations of R₁-R₇ disclosed above are contemplated.

The structures and chemical names of particularly preferred compounds are shown below.

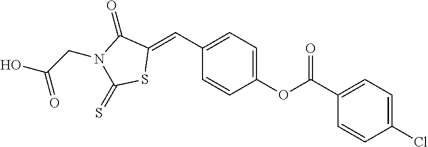 [SMILES: O=C(O)CN1C(=O)/C(=C/c2ccc(OC(=O)c3ccc(Cl)cc3)cc2)SC1=S]

### Compound 2 ((5-{4-[(4-chlorobenzoyl)oxy]benzylidene}-4-oxo-2-thioxo-1,3-thiazolidin-3-yl)acetic acid)

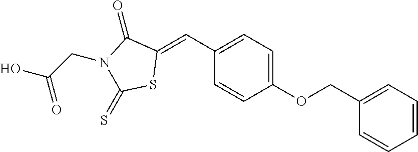 [SMILES: O=C(O)CN1C(=O)/C(=C/c2ccc(OCc3ccccc3)cc2)SC1=S]

### Compound 3 ({5-[4-(benzyloxy)benzylidene]-4-oxo-2-thioxo-1,3-thiazolidin-3-yl}acetic acid)

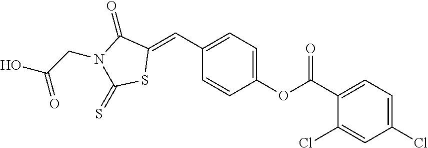 [SMILES: O=C(O)CN1C(=O)/C(=C/c2ccc(OC(=O)c3ccc(Cl)cc3Cl)cc2)SC1=S]

### Compound 4 ((5-{4-[(2,4-dichlorobenzoyl)oxy]benzylidene}-4-oxo-2-thioxo-1, 3-thiazolidin-3-yl)acetic acid)

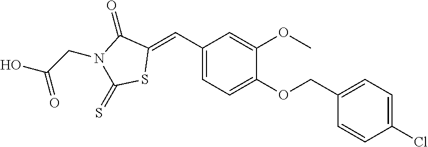 [SMILES: COc1cc(/C=C2\SC(=S)N(CC(=O)O)C2=O)ccc1OCc1ccc(Cl)cc1]

### Compound 6 ((5-{4-[(4-chlorobenzyl)oxy]-3-methoxybenzylidene}-4-oxo-2-thioxo-1,3-thiazolidin-3-yl)acetic acid)

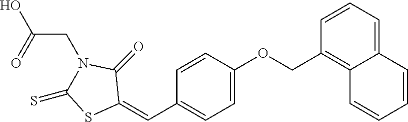 [SMILES: O=C(O)CN1C(=O)/C(=C\c2ccc(OCc3cccc4ccccc34)cc2)SC1=S]

### Compound 7 ({5-[4-(1-naphthylmethoxy)benzylidene]-4-oxo-2-thioxo-1,3-thiazolidin-3-yl}acetic acid)

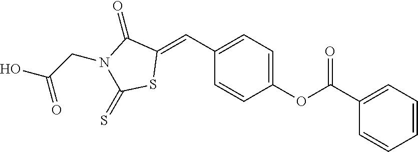 [SMILES: O=C(O)CN1C(=O)/C(=C/c2ccc(OC(=O)c3ccccc3)cc2)SC1=S]

### Compound 10 ({5-[4-(benzoyloxy)benzylidene]-4-oxo-2-thioxo-1,3-thiazolidin-3-yl}acetic acid)

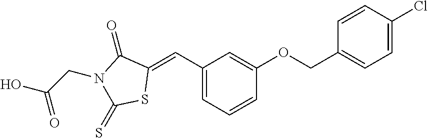 [SMILES: O=C(O)CN1C(=O)/C(=C/c2cccc(OCc3ccc(Cl)cc3)c2)SC1=S]

### Compound 13 ((5-{3-[(4-chlorobenzyl)oxy]benzylidene}-4-oxo-2-thioxo-1,3-thiazolidin-3-yl)acetic acid)

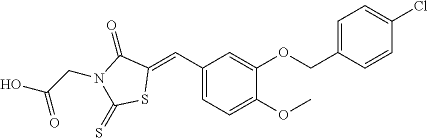 [SMILES: COc1ccc(/C=C2\SC(=S)N(CC(=O)O)C2=O)cc1OCc1ccc(Cl)cc1]

### Compound 15 ((5-{3-[(4-chiorobenzyl)oxy]-4-methoxybenzylidene}-4-oxo-2-thioxo-1,3-thiazolidin-3-yl)acetic acid)

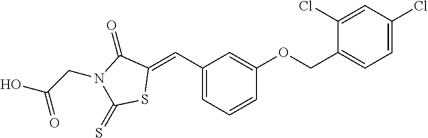 [SMILES: O=C(O)CN1C(=O)/C(=C/c2cccc(OCc3ccc(Cl)cc3Cl)c2)SC1=S]

### Compound 17 ((5-{3-[(2,4-dichlorobenzyl)oxy]benzylidene}-4-oxo-2-thioxo-1,3-thiazolidin-3-yl)acetic acid)

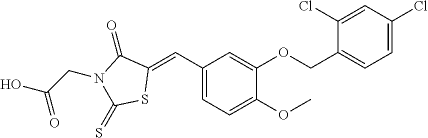 [SMILES: COc1ccc(/C=C2\SC(=S)N(CC(=O)O)C2=O)cc1OCc1ccc(Cl)cc1Cl]

### Compound 18 ((5-{3-[(2,4-dichlorobenzyl)oxy]-4-methoxybenzylidene}-4-oxo-2-thioxo-1,3-yl)acetic acid)

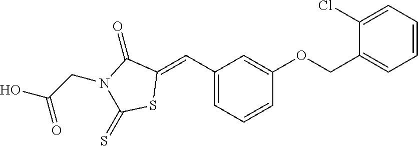 [SMILES: O=C(O)CN1C(=O)/C(=C/c2cccc(OCc3ccccc3Cl)c2)SC1=S]

### Compound 19 ((5-{3-[(2-chlorobenzyl)oxy]benzvlidene}-4-oxo-2-thioxo-1,3-thiazolidin-3-yl)acetic acid)

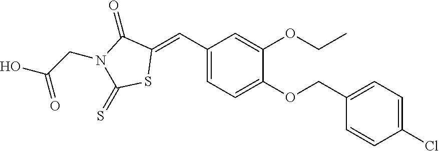 [SMILES: CCOc1cc(/C=C2\SC(=S)N(CC(=O)O)C2=O)ccc1OCc1ccc(Cl)cc1]

### Compound 20 ((5-{4-[(4-chlorobenzyl)oxy]-3-ethoxybenzylidene}-4-oxo-2-thioxo-1,3-thiazolidin-3-yl)acetic acid)

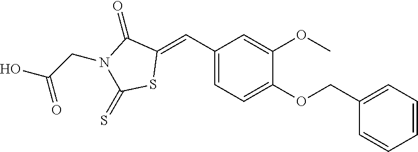 [SMILES: COc1cc(/C=C2\SC(=S)N(CC(=O)O)C2=O)ccc1OCc1ccccc1]

### Compound 22 ({5-[4-(benzyloxy)-3-methoxybenzylidene]-4-oxo-2-thioxo-1,3-thiazolidin-3-yl}acetic acid)

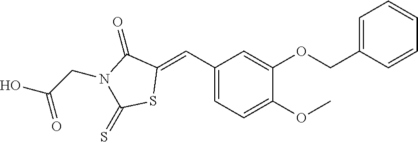 [SMILES: COc1ccc(/C=C2\SC(=S)N(CC(=O)O)C2=O)cc1OCc1ccccc1]

### Compound 24 ({5-[3-benzyloxy)-4-methoxybenzylidene]-4-oxo-2-thioxo-1,3-thiazolidin-3-yl}acetic acid)

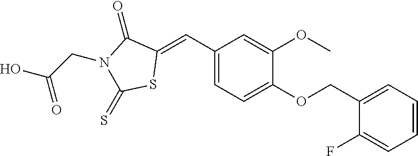 [SMILES: COc1cc(/C=C2\SC(=S)N(CC(=O)O)C2=O)ccc1OCc1ccccc1F]

### Compound 25 ((5-{4-[(2-fluorobenzyl)oxy]-3 -methoxybenzylidene}-4-oxo-2-thioxo1,3-thiazolidin-3-yl)acetic acid)

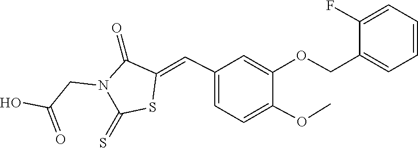 [SMILES: COc1ccc(/C=C2\SC(=S)N(CC(=O)O)C2=O)cc1OCc1ccccc1F]

### Compound 27 ((5-{3-[(2-fluorobenzyl)oxy]-4-methoxybenzylidene}-4-oxo-2-thioxo-1,3-thiazolidin-3-yl)acetic acid)

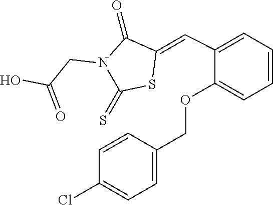 [SMILES: O=C(O)CN1C(=O)/C(=C/c2ccccc2OCc2ccc(Cl)cc2)SC1=S]

### Compound 28 ((5-{2-[(4-chlorobenzyl)oxy]benzylidene}-4-oxo-2-thioxo-1,3-thiazolidin-3-yl)acetic acid)

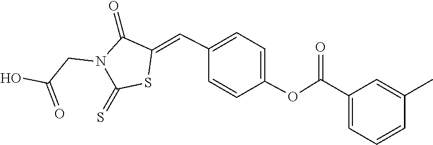 [SMILES: Cc1cccc(C(=O)Oc2ccc(/C=C3\SC(=S)N(CC(=O)O)C3=O)cc2)c1]

### Compound 29 ((5-{4[(3-methylbenzoyl)oxy]benzylidene}-4-oxo-2-thioxo-1,3-thiazolidin-3-yl)acetic acid)

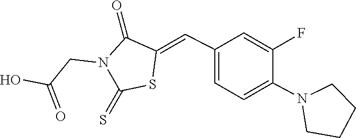 [SMILES: O=C(O)CN1C(=O)/C(=C/c2ccc(N3CCCC3)c(F)c2)SC1=S]

### Compound 30 ({5-[3-fluoro-4-(1-pyrrolidinyl)benzylidene]-4-oxo-2-thioxo-1,3-thiazolidin-3-yl}acetic acid)

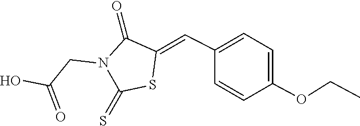 [SMILES: CCOc1ccc(/C=C2\SC(=S)N(CC(=O)O)C2=O)cc1]

### Compound 31 ([5-(4ethoxybenzylidene)-4-oxo-2-thioxo-1,3-thiazolidin-3-yl]acetic acid)

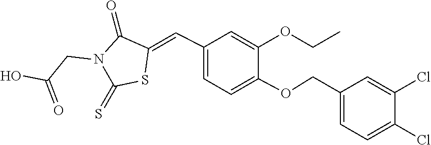 [SMILES: CCOc1cc(/C=C2\SC(=S)N(CC(=O)O)C2=O)ccc1OCc1ccc(Cl)c(Cl)c1]

### Compound 34 ((5 -{4-[(3 ,4-dichlorobenzyl)oxy]-3-ethoxybenzylidene}-4-oxo-2-thioxo-1,3-thiazolidin-3-yl)acetic acid)

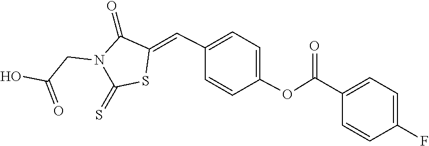 [SMILES: O=C(O)CN1C(=O)/C(=C/c2ccc(OC(=O)c3ccc(F)cc3)cc2)SC1=S]

### Compound 35 ((5-{4-[(4-fluorobenzoyl)oxy]benzylidene}-4-oxo-2-thioxo-1,3-thiazolidin-3-yl)acetic acid)

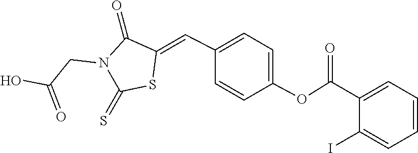 [SMILES: O=C(O)CN1C(=O)/C(=C/c2ccc(OC(=O)c3ccccc3I)cc2)SC1=S]

### Compound 36 ((5-{4-[(2-iodobenzoyl)oxy]benzylidene}-4-oxo-2-thioxo-1,3-thiazolidin-3-yl)acetic acid)

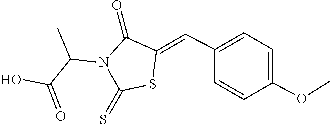 [SMILES: COc1ccc(/C=C2\SC(=S)N(C(C)C(=O)O)C2=O)cc1]

### Compound 37 (2-[5-(4-methoxybenzylidene)-4-oxo-2-thioxo-1,3-thiazolidin-3-yl]propanoic acid)

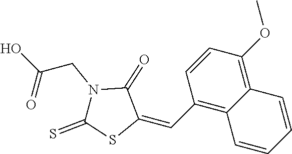 [SMILES: COc1ccc(/C=C2/SC(=S)N(CC(=O)O)C2=O)c2ccccc12]

### Compound 42 ({5-[(4-methoxy-1-naphthyl)methylene]-4-oxo-2-thioxo-1,3-thiazolidin-3-yl}acetic acid)

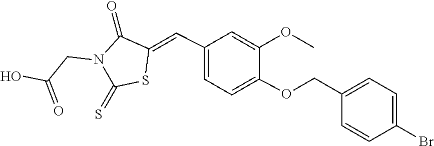 [SMILES: COc1cc(/C=C2\SC(=S)N(CC(=O)O)C2=O)ccc1OCc1ccc(Br)cc1]

### Compound 44 ((5-{4-[(4-bromobenzyl)oxy]-3-methoxybenzylidene}-4-oxo-2-thioxo-1,3-thiazolidin-3-yl)acetic acid)

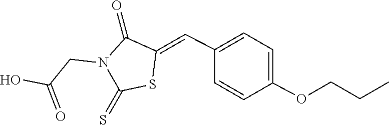 [SMILES: CCCOc1ccc(/C=C2\SC(=S)N(CC(=O)O)C2=O)cc1]

### Compound 48 ([4-oxo-5-(4-propoxybenzylidene)-2-thioxo-1,3-thiazolidin-3-yl]acetic acid)

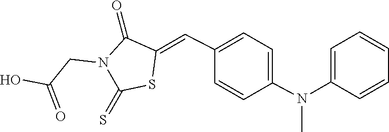 [SMILES: CN(c1ccccc1)c1ccc(/C=C2\SC(=S)N(CC(=O)O)C2=O)cc1]

### Compound 49 ((5-{4-[methyl(phenyl)amino]benzylidene}-4-oxo-2-thioxo-1,3-thiazolidin-3yl) acetic acid)

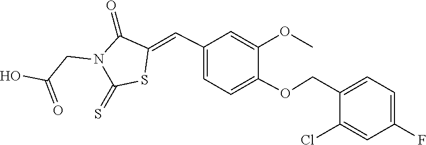 [SMILES: COc1cc(/C=C2\SC(=S)N(CC(=O)O)C2=O)ccc1OCc1ccc(F)cc1Cl]

### Compound 50 ((5-{4-[(2-chloro-4-fluorobenzyl)oxy]-3 -methoxybenzylidene}-4-oxo-2-thioxo-1,3-thiazolidin-3-yl)acetic acid)

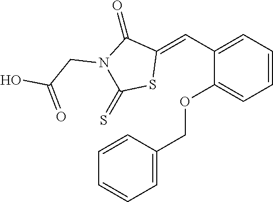 [SMILES: O=C(O)CN1C(=O)/C(=C/c2ccccc2OCc2ccccc2)SC1=S]

### Compound 52 ({5[2-(benzyloxy)benzylidene]4-oxo-2-thioxo-1,3-thiazolidin-3-yl}acetic acid)

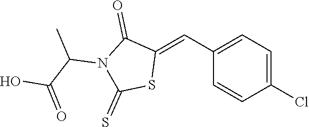 [SMILES: CC(C(=O)O)N1C(=O)/C(=C/c2ccc(Cl)cc2)SC1=S]

### Compound 53 (2-[5-(4-chlorobenzylidene)-4-oxo-2-thioxo-1,3-thiazolidin-3-yl]propanoic acid)

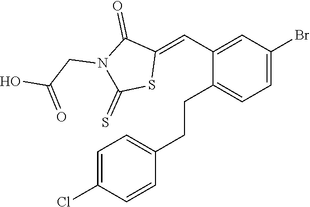 [SMILES: O=C(O)CN1C(=O)/C(=C/c2cc(Br)ccc2CCc2ccc(Cl)cc2)SC1=S]

### Compound 56 ((5-{5-bromo-2-[(4-chlorobenzyl)oxy]benzylidene}-4-oxo-2-thioxo-1,3-thiazolidin-3-yl)acetic acid)

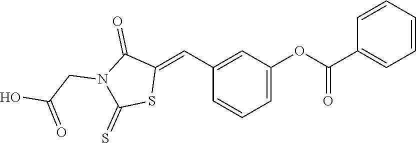 [SMILES: O=C(O)CN1C(=O)/C(=C/c2cccc(OC(=O)c3ccccc3)c2)SC1=S]

### Compound 60 ({5-[3(benzoyloxy)benzylidene]-4-oxo-2-thioxo-1,3-thiazolidin-3-yl}acetic acid)

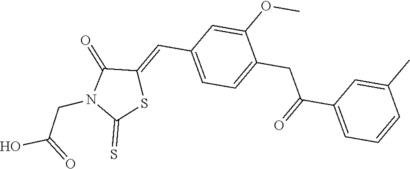 [SMILES: COc1cc(/C=C2\SC(=S)N(CC(=O)O)C2=O)ccc1CC(=O)c1cccc(C)c1]

### Compound 62 ((5-{3-methoxy-4-[(3-methylbenzoyl)oxy]benzylidene}-4-oxo-2-thioxo-1,3-thiazolidin-3-yl)acetic acid)

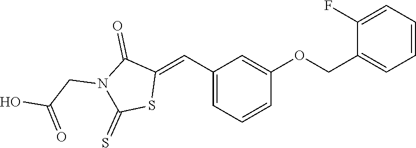 [SMILES: O=C(O)CN1C(=O)/C(=C/c2cccc(OCc3ccccc3F)c2)SC1=S]

### Compound 63 ((5-{3-[(2-fluorobenzyl_oxy]benzylidene}-4-oxo-2-thioxo-1,3-thiazolidin-3-yl)acetic acid)

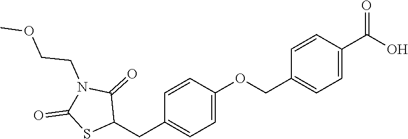 [SMILES: COCCN1C(=O)SC(Cc2ccc(OCc3ccc(C(=O)O)cc3)cc2)C1=O]

### Compound 69 (4-[(4-{[3-(2-methoxyethyl)-4-oxo-2-thioxo-1,3-thiazolidin-5-ylidene]methyl}phenoxy)methyl]benzoic acid)

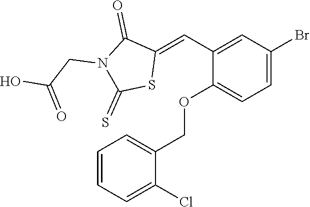 [SMILES: O=C(O)CN1C(=O)/C(=C/c2cc(Br)ccc2OCc2ccccc2Cl)SC1=S]

### Compound 80 ((5-{5-bromo-2-[(2-chlorobenzyl)oxy]benzylidene}-4-oxo-2-thioxo-1,3-thiazolidin-3-yl) acetic acid)

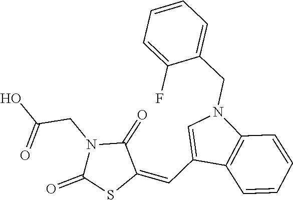 [SMILES: O=C(O)CN1C(=O)S/C(=C/c2cn(Cc3ccccc3F)c3ccccc23)C1=O]

### Compound 81 ((5-{[1-(2-fluorobenzyl)-1H-indol-3-yl]methylene}-4-oxo-2-thioxo-1,3-thiazolidin-3-yl)acetic acid)

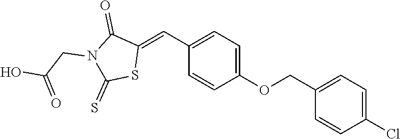 [SMILES: O=C(O)CN1C(=O)/C(=C/c2ccc(OCc3ccc(Cl)cc3)cc2)SC1=S]

### Compound 82 ((5-{4-[(4-chlorobenzyl)oxy]benzylidene}-4-oxo-2-thioxo-1,3-thiazolidin-3-yl) acetic acid)

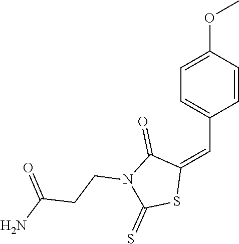 [SMILES: COc1ccc(/C=C2/SC(=S)N(CCC(N)=O)C2=O)cc1]

### Compound 87 (3-[5-(4-methoxybenzylidene)-4-oxo-2-thioxo-1,3-thiazolidin-3-yl]propanamide)

1. Pharmaceutically Acceptable Salts

Also disclosed herein are pharmaceutically acceptable salts of the compound represented by Formula I, and the derivatives thereof as well as other compounds that were determined to bind DHFR. Examples of pharmaceutically acceptable salts include, but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

2. Intermediates

The compounds identified herein using virtual ligand screening can be used as a starting compound or intermediate compound to produce a final compound that has increased binding to an enzyme, for example DHFR, or increased inhibition of the enzyme, for example DHFR, relative to starting compound or intermediate compound. The compounds identified herein can be modified to increase bioavailability, increase half-life in the blood stream, increase solubility, increase hydrophilicity, increase hydrophobicity, or a combination thereof using conventional techniques. The compounds can be modified to incorporate polar functional groups, such as the alcohol, amine, amide, carboxylic acid, sulfonic acid and phosphate groups, which either ionize or are capable of relatively strong intermolecular forces of attraction with water (hydrogen bonding), usually resulting in analogues with an increased water solubility. Acidic and basic groups are particularly useful, since these groups can be used to form salts, which would give a wider range of dosage forms for the final product. However, the formation of zwitterions by the introduction of either an acid group into a structure containing a base or a base group into a structure containing an acid group can reduce water solubility. Introduction of weakly polar groups, such as carboxylic acid esters, aryl halides and alkyl halides, will not significantly improve water solubility and can result in enhanced lipid solubility.

The incorporation of acidic residues into the compound is less likely to change the type of activity, but it can result in the analogue exhibiting hemolytic properties. Furthermore, the introduction of an aromatic acid group usually results in anti-inflammatory activity, whilst carboxylic acids with an alpha functional group may act as chelating agents. Basic water solubilizing groups have a tendency to change the mode of action, since bases often interfere with neurotransmitters and biological processes involving amines. However, their incorporation does mean that the analogue can be formulated as a wide variety of acid salts. Non-ionizable groups do not have the disadvantages of acidic and basic groups.

Groups that are bound directly to the carbon skeleton of the lead compound by less reactive C—C, C—O and C—N bonds are likely to be irreversibly attached to the lead structure. Groups that are linked to the compound by ester, amide, phosphate, sulfate and glycosidic bonds are more likely to be metabolized from the resulting analogue to reform the parent compound as the analogue is transferred from its point of administration to its site of action. Compounds with this type of solubilizing group are acting as prodrugs and so their activity is more likely to be the same as the parent compound. However, the rate of loss of the solubilizing group will depend on the nature of the transfer route, and this could affect the activity of the drug.

To preserve the type of activity exhibited by the compound, a water solubilizing group should be attached to a part of the structure that is not involved in the drug—receptor interaction. Consequently, the route used to introduce a new water solubilizing group and its position in the lead compound will depend on the relative reactivities of the compound and the rest of the molecule. Examples of water solubilizing structures and the routes used to introduce them into the lead structures. O—alkylation, N—alkylation, O—acylation and N—acylation reactions are used to introduce both acidic and basic groups. Acetylation methods use both the appropriate acid chloride and anhydride.

Examples of water solubilizing structures and the routes used to introduce them into lead compounds include but are not limited to phosphate acid halides for introducing phosphate groups into compounds. Structures containing hydroxy groups have been introduced by reaction of the corresponding monochlorinated hydrin and the use of suitable epoxides. Sulphonic acid groups may be introduced by either direct sulfonation or the addition of bisulfate to reactive C═C bonds.

B. Other Bioactive Agents

Other bioactive agents that can be used with the compounds disclosed herein include, but are not limited to antimicrobial agents, in particular antibiotics, disinfectants, anti-scarring agents and anti-inflammatory agents.

Antimicrobial agents that may be incorporated into the perforated collagen coated meshes, include, but are not limited to, antibacterial drugs, antiviral agents, antifungal agents, and antiparisitic drugs. Antimicrobial agents include substances that kill or inhibit the growth of microbes such as microbicidal and microbiostatic agents. Antimicrobial agents that may be incorporated into the perforated collagen coated meshes, include, but are not limited to: rifampin; minocycline and its hydrochloride, sulfate, or phosphate salt; triclosan; chlorhexidine; vancomycin and its hydrochloride, sulfate, or phosphate salt; tetracycline and its hydrochloride, sulfate, or phosphate salt, and derivatives; gentamycin; cephalosporin antimicrobials; aztreonam; cefotetan and its disodium salt; loracarbef; cefoxitin and its sodium salt; cefazolin and its sodium salt; cefaclor; ceftibuten and its sodium salt; ceftizoxime; ceftizoxime sodium salt; cefoperazone and its sodium salt; cefuroxime and its sodium salt; cefuroxime axetil; cefprozil; ceftazidime; cefotaxime and its sodium salt; cefadroxil; ceftazidime and its sodium salt; cephalexin; cefamandole nafate; cefepime and its hydrochloride, sulfate, and phosphate salt; cefdinir and its sodium salt; ceftriaxone and its sodium salt; cefixime and its sodium salt; cefpodoxime proxetil; meropenem and its sodium salt; imipenem and its sodium salt; cilastatin and its sodium salt; azithromycin; clarithromycin; dirithromycin; erythromycin and hydrochloride, sulfate, or phosphate salts, ethylsuccinate, and stearate forms thereof, clindamycin; clindamycin hydrochloride, sulfate, or phosphate salt; lincomycin and hydrochloride, sulfate, or phosphate salt thereof, tobramycin and its hydrochloride, sulfate, or phosphate salt; streptomycin and its hydrochloride, sulfate, or phosphate salt; neomycin and its hydrochloride, sulfate, or phosphate salt; acetyl sulfisoxazole; colistimethate and its sodium salt; quinupristin; dalfopristin; amoxicillin; ampicillin and its sodium salt; clavulanic acid and its sodium or potassium salt; penicillin G; penicillin G benzathine, or procaine salt; penicillin G sodium or potassium salt; carbenicillin and its disodium or indanyl disodium salt; piperacillin and its sodium salt; ticarcillin and its disodium salt; sulbactam and its sodium salt; moxifloxacin; ciprofloxacin; ofloxacin; levofloxacins; norfloxacin; gatifloxacin; trovafloxacin mesylate; alatrofloxacin mesylate; trimethoprim; sulfamethoxazole; demeclocycline and its hydrochloride, sulfate, or phosphate salt; doxycycline and its hydrochloride, sulfate, or phosphate salt; oxytetracycline and its hydrochloride, sulfate, or phosphate salt; chlortetracycline and its hydrochloride, sulfate, or phosphate salt; metronidazole; dapsone; atovaquone; rifabutin; linezolide; polymyxin B and its hydrochloride, sulfate, or phosphate salt; sulfacetamide and its sodium salt; clarithromycin; gentamicin; biguanide; bacitracin; silver, copper, zinc, and gold ions, salts, and complexes. In a preferred form the antimicrobial agents incorporated into the implants are (i) rifampin and (ii) minocycline and its hydrochloride, sulfate, or phosphate salt. In a particularly preferred form, the perforated collagen coated meshes comprise rifampin and minocycline or its hydrochloride, sulfate, or phosphate salt.

C. Formulations

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions.

The compounds disclosed herein can also be formulated for use as a disinfectant, for example, in a hospital environment.

These compositions can take the form of solutions, suspensions, emulsion, tablets, pills, capsules, powders, sustained-release formulations and the like.

Suitable pharmaceutical excipients include starch, glucose, sucrose, gelatin, lactose, malt, rice, flour, chalk, silica gel, sodium stearate, glycerol monostearate, talc, sodium chloride, glycerol, propylene, glycol, water, ethanol and the like. The pharmaceutical composition may also contain wetting or emulsifying agents or suspending/diluting agents, or pH buffering agents, or agents for modifying or maintaining the rate of release of the compounds/antibiotics, all of which are disclosed further herein.

2. Parenteral Formulations

The compounds described herein can be formulated for parenteral administration. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

If for intravenous administration, the compositions are packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection. The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine, and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

(a) Controlled Release Formulations

The parenteral formulations described herein can be formulated for controlled release including immediate release, delayed release, extended release, pulsatile release, and combinations thereof

3. Nano- and microparticles

For parenteral administration, the one or more compounds, and optional one or more additional active agents, can be incorporated into microparticles, nanoparticles, or combinations thereof that provide controlled release of the compounds and/or one or more additional active agents. In forms wherein the formulations contain two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

2. Method of Making Nano- and Microparticles Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some forms, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some forms, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

(b) Injectable/Implantable Formulations

The compounds described herein can be incorporated into injectable/implantable solid or semi-solid implants, such as polymeric implants. In some forms, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

2. Enteral Formulations

Oral formulations can include standard carriers such as pharmaceutical grades of mannitol, lactose, sodium saccharine, starch, magnesium stearate, cellulose, magnesium carbonate, etc. Such compositions will contain a therapeutically effective amount of the compound and/or antibiotic together with a suitable amount of carrier so as to provide the proper form to the patient based on the mode of administration to be used. Suitable oral dosage forms include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof. Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. “Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. “Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

(a) Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another form, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another form, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

(1) Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred forms, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred forms, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred form, the acrylic polymer is an acrylic resin lacquer such as that which is commercially available from Rohm Pharma under the tradename EUDRAGIT®. In further preferred forms, the acrylic polymer comprises a mixture of two acrylic resin lacquers commercially available from Rohm Pharma under the tradenames EUDRAGIT® RL30D and EUDRAGIT RS30D, respectively. EUDRAGIT® RL30D and EUDRAGIT RS30D are copolymers of acrylic and methacrylic esters with a low content of quaternary ammonium groups, the molar ratio of ammonium groups to the remaining neutral (meth)acrylic esters being 1:20 in EUDRAGIT RL30D and 1:40 in EUDRAGIT® RS30D. The mean molecular weight is about 150,000. EUDRAGIT ® S-100 and EUDRAGIT ® L-100 are also preferred. The code designations RL (high permeability) and RS (low permeability) refer to the permeability properties of these agents. EUDRAGIT ® RL/RS mixtures are insoluble in water and in digestive fluids. However, multiparticulate systems formed to include the same are swellable and permeable in aqueous solutions and digestive fluids.

The polymers described above such as EUDRAGIT ® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

(2) Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

3. Topical Formulations

Suitable dosage forms for topical administration include creams, ointments, salves, sprays, gels, lotions, emulsions, and transdermal patches. The formulation may be formulated for transmucosal, transepithelial, transendothelial, or transdermal administration. The compounds can also be formulated for intranasal delivery, pulmonary delivery, or inhalation. The compositions may further contain one or more chemical penetration enhancers, membrane permeability agents, membrane transport agents, emollients, surfactants, stabilizers, buffers, and combination thereof.

In certain forms, it may be desirable to provide continuous delivery of one or more compounds to a patient in need thereof. For topical applications, repeated application can be done or a patch can be used to provide continuous administration of the compounds over an extended period of time.

“Buffers” are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred form, the buffer is triethanolamine. “Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In some forms, the emollients are ethylhexylstearate and ethylhexyl palmitate.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In some forms, the emulsifier is glycerol stearate.

“Penetration enhancers” are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

“Preservatives” can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In some forms, the non-ionic surfactant is stearyl alcohol.

(b) Emulsions

An emulsion is a preparation of one liquid distributed in small globules throughout the body of a second liquid. In particular forms, the non-miscible components of the emulsion include a lipophilic component and an aqueous component. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

(b) Lotions

A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In some forms, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

(c) Creams

Creams may contain emulsifying agents and/or other stabilizing agents. In some forms, the formulation is in the form of a cream having a viscosity of greater than 1000 centistokes, typically in the range of 20,000-50,000 centistokes. Creams are often time preferred over ointments, as they are generally easier to spread and easier to remove. The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

(d) Ointments

Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

(e) Gels

Gels are semisolid systems containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

(f) Foams

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases, which can produce flammable or explosive vapors during spraying. Furthermore, the compositions preferably contain no volatile alcohols, which can produce flammable or explosive vapors during use.

4. Disinfecting Formulations

The compounds can be formulated into cleaning formulations. The cleaning formulations are highly efficacious for household cleaning applications (e.g., hard surfaces like floors, countertops, tubs, dishes and softer cloth materials like clothing, sponges, paper towels, etc.), personal care applications (e.g., lotions, shower gels, soaps, shampoos, wipes) and industrial and hospital applications (e.g., sterilization of instruments, medical devices, gloves). These formulations are efficacious for cleaning surfaces which are infected or contaminated with Gram negative bacteria and Gram positive bacteria.

The compounds disclosed herein can be formulated into a solution in a suitable solvent for administration in a spray bottle, the compounds can be formulation as an aerosol or a foam, suitable for spraying onto surfaces, of imbibed into a suitable cloth suitable for wiping down a surface to be disinfected. Methods for making formulations for use as a disinfectant in the forms disclosed herein are known in the art.

Some forms provide NSC309401 or a derivative thereof in a composition containing a pH dye indicator and an alkaline substance. The pH indicator dye indicates what surface has been disinfected and ensures that a sufficient time has passed to disinfect the surface. See for example, U.S. Publication No. 20140057987, which is incorporated by reference in its entirety.

Cleaning formulations can include the compounds and an acceptable carrier. The carrier can be in a wide variety of forms. For example, the carrier may be an aqueous-based solution or cleanser, an alcohol-based solution or gel or an emulsion carrier, including, but not limited to, oil-in-water, water-in-oil, water-in-oil-in-water, and oil-in-water-in-silicone emulsions. The carrier solution containing the compound(s) can be applied directly to the surface to be treated or delivered via a suitable substrate.

The cleaning formulations can be formulated for use on the skin. In these forms the compounds can be formulate in a dermatologically acceptable carrier. The dermatologically acceptable carriers can also be, for example, formulated as alcohol or water based hand cleansers, toilet bars, liquid soaps, shampoos, bath gels, hair conditioners, hair tonics, pastes, or mousses.

Cleaning formulations can contain one or more surfactants. The surfactant is suitably selected from anionic, nonionic, zwitterionic, amphoteric and ampholytic surfactants, as well as mixtures of these surfactants. Such surfactants are well known to those skilled in the detergency art. Nonlimiting examples of possible surfactants include isoceteth-20, sodium methyl cocoyl taurate, sodium methyl oleoyl taurate, and sodium lauryl sulfate. Examples of a broad variety of additional surfactants are described in McCutcheon's Detergents and Emulsifiers. North American Edition (1986), published by Allured Publishing Corporation. The cleansing formulations can optionally contain, at their art-established levels, other materials which are conventionally used in cleansing formulations.

Additional carriers suitable for the cleaning formulations may include various substrate-based products. In such instances, the present formulations may be impregnated into or onto the substrate products and may be allowed to remain wet or may be subjected to a drying process. For instance, suitable carriers include, but are not limited to, dry and wet wipes suitable for personal care and household use (e.g., nonwoven baby wipes, household cleaning wipes, surgical preparation wipes, etc.); diapers; infant changing pads; dental floss; personal care and household care sponges or woven cloths (e.g., washcloths, towels, etc.); tissue-type products (e.g. facial tissue, paper towels, etc.); and disposable garments (e.g., gloves, smocks, surgical masks, infant bibs, socks, shoe inserts, etc.). Cleaning formulations can be incorporated into various household care products including, but not limited to, hard surface cleaners (e.g., disinfectant sprays, liquids, or powders); dish or laundry detergents (liquid or solid), floor waxes, glass cleaners, etc.

Exemplary carriers can include aqueous solutions, e.g., having from about 0% to about 98.8%, by weight of the composition, of water. Additionally, carriers may contain an aqueous alcohol solution. The amount of alcohol present in the alcohol solution will vary depending on the type of product in which the composition is incorporated, i.e., say a wipe where the preferred amount of alcohol present would be from about 0% to about 25% whereas a hand sanitizer preferably contains from about 60% to about 95%, of alcohol. Therefore, suitable dermatologically acceptable alcohol solutions or gels may contain from about 0% to about 95%, by weight of the composition, of an alcohol.

Alcohols suitable for inclusion in the alcohol solutions of the carrier include, but are not limited to, monohydric alcohols, dihydric alcohols, and combinations thereof. More preferred alcohols are selected from the group consisting of monohydric linear or branched C2-C18 alcohols. The most preferred alcohols are selected from the group consisting of ethanol, isopropanol, n—propanol, butanol, and combinations thereof. The cleaning formulations which contain an alcohol solution may be anhydrous or water containing.

Thickeners can be added to the water or alcohol based to form a gel. Examples of suitable thickeners include, but are not limited to, naturally-occurring polymeric materials such as sodium alginate, xanthan gum, quince seed extract, tragacanth gum, starch and the like, semi-synthetic polymeric materials such as cellulose ethers (e.g. hydroxyethyl cellulose, methyl cellulose, carboxymethyl cellulose, hydroxy propylmethyl cellulose), polyvinylpyrrolidone, polyvinyl alcohol, guar gum, hydroxypropyl guar gum, soluble starch, cationic celluloses, cationic guars and the like and synthetic polymeric materials such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like. Inorganic thickeners may also be used such as aluminum silicates, such as, for example, bentonites, or a mixture of polyethylene glycol and polyethylene glycol stearate or distearate.

The cleaning formulations can contain, in addition to the compounds described above, one or more antimicrobial or antifungal agents. Such agents are capable of destroying microbes, preventing the development of microbes or preventing the pathogenic action of microbes. Examples of additional antimicrobial and antifungal agents include β-lactam drugs, quinolone drugs, ciprofloxacin, norfloxacin, tetracycline, erythromycin, amikacin, 2,4,4′-trichloro-2′-hydroxy diphenyl ether (Triclosan®), phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, pyrithiones (especially zinc pyrithione which is also known as ZPT), dimethyldimethylol hydantoin (Glydant®), methylchloroisothiazolinone/methylisothiazolinone (Kathon CG®), sodium sulfite, sodium bisulfite, imidazolidinyl urea (Germall 115®), diazolidinyl urea (Germaill II®), benzyl alcohol, 2-bromo-2-nitropropane-1,3 -diol (Bronopol®), formalin (formaldehyde), iodopropenyl butylcarbamate (Polyphase P100®), chloroacetamide, methanamine, methyldibromonitrile glutaronitrile (1,2-Dibromo-2,4-dicyanobutane or Tektamer®), glutaraldehyde, 5-bromo-5-nitro-1,3 -dioxane (Bronidox®), phenethyl alcohol, o-phenylphenol/sodium o-phenylphenol, sodium hydroxymethylglycinate (Suttocide A®), polymethoxy bicyclic oxazolidine (Nuosept C®), dimethoxane, thimersal dichlorobenzyl alcohol, captan, chloyhenenesin, dichlorophene, chlorbutanol, glyceryl laurate, halogenated diphenyl ethers like 2,4,4′-trichloro-2′-hydroxy-diphenyl ether (Triclosan® or TCS), 2,2′-dihydroxy-5,5′-dibromo-diphenyl ether, phenolic compounds like phenol, 2-methyl phenol, 3-methyl phenol, 4-methyl phenol, 4-ethyl phenol, 2,4-dimethyl phenol, 2,5-dimethyl pPhenol, 3,4-dimethyl phenol, 2,6-dimethyl phenol, 4-n-propyl phenol, 4-n-butyl phenol, 4-n-amyl phenol, 4-tert-amyl phenol, 4-n-hexyl phenol, 4-n-heptyl phenol, mono-and poly-alkyl and aromatic halophenols such as p-chlorophenol, methyl p-chlorophenol, ethyl p-chlorophenol, n-propyl p-chlorophenol, n-butyl p-chlorophenol, n-amyl p-chlorophenol, sec-amyl p-chlorophenol, n-hexyl p-chlorophenol, cyclohexyl p-chlorophenol, n-heptyl p-chlorophenol, n-octyl p-chlorophenol, o-chlorophenol, methyl o-chlorophenol, ethyl o-chlorophenol, n-propyl o-chlorophenol, n-butyl o-chlorophenol, n-amyl o-chlorophenol, tert-amyl o-chlorophenol, n-hexyl o-chlorophenol, n-heptyl o-chlorophenol, o-benzyl p-chlorophenol, o-benzyl-m-methyl p-chlorophenol, o-benzyl-m, m-dimethyl p-chlorophenol, o-phenylethyl p-chlorophenol, o-phenylethyl-m-methyl p-chlorophenol, 3-methyl p-chlorophenol, 3,5-dimethyl p-chlorophenol, 6-ethy 1-3 -methyl p-chlorophenol, 6-n-propyl-3-methyl p-chlorophenol, 6-iso-propyl-3 -methyl p-chlorophenol, 2-ethyl-3,5-dimethyl p-chlorophenol, 6-sec-butyl-3-methyl p-chlorophenol, 2-iso-propyl-3,5 -dimethyl p-chlorophenol, 6-diethylmethyl-3-methyl p-chlorophenol, 6-iso-propyl-2-ethyl-3 -methyl p-chlorophenol, 2-sec-amyl-3,5-dimethyl p-chlorophenol, 2-diethylmethyl-3 ,5-dimethyl p-chlorophenol, 6-sec-octyl-3 -methyl p-chlorophenol, p-chloro-m-cresol, p-bromophenol, methyl p-bromophenol, ethyl p-bromophenol, n-propyl p-bromophenol, n-butyl p-bromophenol, n-amyl p-bromophenol, sec-amyl p-bromophenol, n-hexyl p-bromophenol, cyclohexyl p-bromophenol, o-bromophenol, tert-amyl o-bromophenol, n-hexyl o-bromophenol, n-propyl-m,m-dimethyl o-bromophenol, 2-phenyl phenol, 4-chloro-2-methyl phenol, 4-chloro-3 -methyl phenol, 4- chloro-3, 5-dimethyl phenol, 2,4-dichloro-3,5-dimethylphenol, 3 ,4,5,6-terabromo-2-methylphenol, 5-methyl-2-pentylphenol, 4-isopropyl-3-methylphenol, para-chloro-meta-xylenol (PCMX), chlorothymol, 5-chloro-2-hydroxydiphenylmethane, resorcinol and its derivatives including methyl resorcinol, ethyl resorcinol, n-propyl resorcinol, n-butyl resorcinol, n-amyl resorcinol, n-hexyl resorcinol, n-heptyl resorcinol, n-octyl resorcinol, n-nonyl resorcinol, phenyl resorcinol, benzyl resorcinol, phenylethyl resorcinol, phenylpropyl resorcinol, p-chlorobenzyl resorcinol, 5-chloro 2,4-dihydroxydiphenyl methane, 4′-chloro 2,4-dihydroxydiphenyl methane, 5-bromo 2,4-dihydroxydiphenyl methane, and 4′ -bromo 2,4-dihydroxydiphenyl methane, bisphenolic compounds like 2,2′-methylene bis (4-chlorophenol), 2,2′-methylene bis (3,4,6-trichlorophenol), 2,2′-methylene bis (4-chloro-6-bromophenol), bis (2-hydroxy-3,5-dichlorophenyl) sulphide, and bis (2-hydroxy-5-chlorobenzyl)sulphide, benzoic esters (parabens) like methylparaben, propylparaben, butylparaben, ethylparaben, isopropylparaben, isobutylparaben, benzylparaben, sodium methylparaben, and sodium propylparaben, halogenated carbanilides (e.g., 3,4,4′-trichlorocarbanilides (Triclocarban®or TCC), 3 -trifluoromethyl-4,4′-dichlorocarbanilide, 3,3′,4-trichlorocarbanilide, etc.), cationic actives such as benzalkonium chloride, and clotrimazole. Another class of antimicrobial agents (specifically antibacterial agents) which are useful in the disclosed compositions and methods are the so-called “natural” antibacterial actives, referred to as natural essential oils. Typical natural essential oil antibacterial actives include oils of anise, lemon, orange, rosemary, wintergreen, thyme, lavender, cloves, hops, tea tree, citronella, wheat, barley, lemongrass, cedar leaf, cedarwood, cinnamon, fleagrass, geranium, sandalwood, violet, cranberry, eucalyptus, vervain, peppermint, gum benzoin, basil, fennel, fir, balsam, menthol, ocmea origanum, Hydastis carradensis, Berberidaceae daceae, Ratanhiae and Curcuma longa.

The cleaning formulations may be packaged in a variety of suitable packaging known to those skilled in the art. The liquid formulations may desirably be packaged in manually operated spray dispensing containers, which are usually made of synthetic organic polymeric plastic materials. Accordingly, disinfecting formulations containing the compounds described herein and packaged in a spray dispenser, preferably in a trigger spray dispenser or a pump spray dispenser, are envisioned. Spray-type dispensers permit uniform application to a relatively large area of a surface to be disinfected with liquid cleaning formulations described herein.

The disclosed compounds, for example NSC309401 or a derivative thereof can be impregnated into a nonwoven absorbent wipe. Disinfectant wet wipes are also disclosed for example in U.S. Pat. No. 8,563,017. The disclosed compounds, for example NSC309401 or a derivative thereof can be in an aqueous foam with a special surfactant system capable of generating a foam. See U.S. Pat. Nos. 8,097,265, 5,891,922 and 4,889,645.

The disclosed compounds, for example NSC309401 or a derivative thereof can be in a pressurized spray aerosol. See also, U.S. Publication No. 20010053333 which discloses a liquid flash-dry aerosol disinfectant composition with a flash vaporization component and an effective amount of an antimicrobial agent.

It is within the abilities of one of ordinary skill in the art to determine the effective amount of the compounds disclosed herein to include in an aerosol, foam, solution or disinfectant cloth for the purpose of sterilizing for example, high risk hospital surfaces.

### III. Methods of Use

The methods disclosed herein use two approaches to counter bacterial resistance to antibacterials. First, the methods use the compounds disclosed herein in combination with existing antibiotics. This strategy increases the hurdles for the bacteria to acquire the required resistance factors to survive, simultaneously slowing down the rise of the resistant strains. The methods described herein preserve the use of the existing antibiotics, which will prolong the clinical life span of the use of those antibiotics, providing time for development of a new generation of antibiotics. Not only does this combination treatment mount a greater hurdle for emergence of resistance strains, the compounds disclosed herein potentiate the effect of the co-administered antibiotic. A second approach is targeting virulence factors of the bacteria. Since virulence factors are auxiliary genes for the bacterial survival, having those genes inactivated by chemical genetics methods will not pose direct growth challenges for the bacteria. This in return will reduce the likelihood of developing an escape mutant, therefore reducing the development of resistant strains.

Accordingly, some forms disclose methods of treating or preventing microbial infection, such as but not limited to, Staphyloccous aureus, Pseudomonas aeruginosa, Listeria monocytogenes, Burkholderia cepacia, Escherichia coli, Enterococcus faecalis, Streptococcus pneumoniae infection, in a subject, by administering an effective amount of one or more compound according to any of formulas (Compound 2-compound 82) to a subject in need thereof. In some forms, the methods further comprise administering an effective amount of at least one antibiotic, such as, e.g., gentamicin, amikacin, kanamycin, neomycin, spectinomycin, neamine or any combination thereof. The antibiotic can be administered at the same time, before, or after the compound administration. Further, the administration of the antibiotic and disclosed compound(s) can be concurrently in the form of an administered pharmaceutical composition.

Administration may be locally (confined to a single cell or tissue) and/or systemically in the subject. It may be desirable to administer the compounds/antibiotics and disclosed pharmaceutical compositions locally to the area in need of treatment, such as areas of infection. This method of administration may be achieved by, for example, and not by way of limitation, local infusion during surgery, topical application (e.g., in conjunction with a wound dressing after surgery), by injection, by catheter, or by means of an implant (e.g., a porous membrane).

In some forms, the compounds/antibiotics or pharmaceutical composition can be delivered in a controlled release system. Such methods may include the use of a pump for administration (e.g., use of an intravenous drip). In another form, a controlled release system can be placed in the proximity of the therapeutic target (e.g., site of infection), requiring only a fraction of the dose required if dosed systemically.

In other forms the disclosed small molecules serve as lead compounds for further modifications to create more potent and specific treatments.

A. Microbes

Exemplary microbes that can be inhibited with the disclosed compounds include, but are not limited to, Staphylococcus aureus, Pseudomonas aeruginosa, Burkholderia cepacia Enterococcus faecalis, Enterococcus faecium and Streptococcus pneumoniae. Preferred forms include methicillin-resistant strains of Staphylococcus aureus and multidrug resistant Listeria monocytogenes, Pseudomonas aeruginosa, Burkholderia cepacia, Enterococcus faecalis, Streptococcus pneumoniae.

Other bacteria that can be treated include, but are not limited to Acinteobacter, Antinomies, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Enterobacter, Enterococcus, Escherichia, Francisella, Halobacterium, Heliobacter, Haemophilus, Hemophilus influenza type B (HIB), Hyphomicrobium, Klebsiella, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

In one preferred form the compositions disclosed herein are used as a topical antibacterial medication for skin infections caused by methicillin-resistant Staphylococcus aureus. Methicillin-resistant Staphylococcus aureus (MRSA) is a type of bacteria that is resistant to many antibiotics. The CDC encourages clinicians to consider MRSA in the differential diagnosis of skin and soft tissue infections (SSTIs) compatible with S. aureus infections, especially those that are purulent (fluctuant or palpable fluid-filled cavity, yellow or white center, central point or “head,” draining pus, or possible to aspirate pus with needle or syringe). A patient's presenting complaint of “spider bite” should raise suspicion of an S. aureus infection. In the community, most MRSA infections are skin infections. In other forms, the compositions can be used to treat systemic infections of lungs or blood caused by methicillin-resistant Staphylococcus aureus in hospitalized or immunocompromised patients. In medical facilities, MRSA causes life-threatening bloodstream infections, pneumonia and surgical site infections.

The spectrum of disease caused by MRSA appears to be similar to that of Staphylococcus aureus in the community. Soft tissue infections (SSTIs), specifically furuncles (abscessed hair follicles or “boils”), carbuncles (coalesced masses of furuncles), and abscesses, are the most frequently reported clinical manifestations. The most common manifestations of community associated-MRSA are simple skin infections, such as impetigo, boils, abscesses, folliculitis, and cellulitis. Rarer, but more serious manifestations can occur, such as necrotizing fasciitis and pyomyositis (most commonly found in the tropics), necrotizing pneumonia, infective endocarditis (which affects the valves of the heart), and bone and joint infections. Additional conditions include severe or extensive disease (e.g., involving multiple sites of infection) or rapid progression in presence of associated cellulitis, signs and symptoms of systemic illness, associated comorbidities or immunosuppression, extremes of age, abscess in an area difficult to drain (e.g., face, hand, and genitalia), associated septic phlebitis, and lack of response to incision and drainage alone, purulent cellulitis, hospitalized patients with complicated SSTI (cSSTI; defined as patients with deeper soft-tissue infections, surgical/traumatic wound infection, and infected ulcers and burns), osteomyelitis, device-related osteoarticular infections, children with minor skin infections (such as impetigo) and secondarily infected skin lesions (such as eczema, ulcers, or lacerations). The compositions disclosed herein can also be used to treat MRSA infections of the CNS, which include, but are not limited to Meningitis, Brain abscess, subdural empyema, spinal epidural abscess (reviewed in Liu, et al., Clin Infect Dis., 52(3):e18-55 (2011)).

The compositions disclosed herein can also be used to treat infections cause by strains of Escherichia coli (drug resistant or otherwise) in subjects especially in immunocompromised patients. Escherichia coli is one of the most frequent causes of many common bacterial infections, including cholecystitis, bacteremia, cholangitis, urinary tract infections other clinical infections such as neonatal meningitis and pneumonia. For example, the compositions can be used treat conditions caused by community- and/or hospital-acquired urinary tract infections (UTI's) caused by strains of Escherichia coli (drug resistant or otherwise) in immunocompromised patients.

Other antibiotic-resistant microorganisms that can be targeted with the compounds disclosed herein include Streptococcus pneumoniae, Campylobacter, Neisseria gonorrhoeae, Salmonella (including drug-resistant non-typhoidal Salmonella and drug-resistant Salmonella serotype typhi), Shigella, Vancomycin-resistant Enterococcus (VRE), Vancomycin-resistant Staphylococcus aureus (VRSA), Erythromycin-resistant Group A Streptococcus, Clindamycin-resistant Group B Streptococcus, Carbapenem-resistant Enterobacteriaceae (CRE), drug-resistant tuberculosis, Extended spectrum Enterobacteriaceae (ESBL), multidrug-resistant Acinetobacter (including MRAB), Clostridium difficile, Enteropathogenic E. coli (EPEC), Pseudomonas aeruginosa, H. pylori, Streptococcus anginosus and Uropathogenic E. coli (UPEC).

B. Method of Treatment 1. Infections

The disclosed compounds and derivatives thereof and combinations thereof can be administered as described above in an amount effective to ameliorate or treat symptoms resulting from infections caused by microbes discussed above, infecting a subject, preferably a human subject. The infection can be systemic or local. The infection can be an infection of the skin, eye, throat, nose, or ear, or of any organ including but not limited to lungs, intestine, heart, brain, and liver. The infection can be an abscess or a wound.

In certain forms, it may be desirable to provide continuous delivery of one or more compounds to a patient in need thereof. For intravenous or intra-arterial routes, this can be accomplished using drip systems, such as by intravenous administration. For topical applications, repeated application can be done or a patch can be used to provide continuous administration of the compounds over an extended period of time.

Because of the potentiating effect of the disclosed compounds, in some form, the amount of a separately, simultaneously, or sequentially administered one or more antimicrobial agents (including one or more antibiotics) may be less than a therapeutically effective or therapeutically optimal dose of the one or more antimicrobial agents (including one or more antibiotics) when administered to the patient or animal that is not in receipt of the disclosed compounds. In some forms, administered one or more antimicrobial agents may be 5%, 10%, 15%, 20%, 30%, 40%, 50%, 60%, 70%, 80% or more, less than a therapeutically effective or therapeutically optimal dose of the one or more antibiotics when administered to the patient or animal that is not in receipt of the disclosed compounds. In another form, the treatment duration of the patient receiving the treatment or prophylaxis of the second form may be 5%, 10%, 15%, 20%, 30%, 40%, 50%, 60%, 70%, 80% or more, less than the treatment duration required when the patient or animal is not in receipt of the disclosed compound.

2. Use as a Sterilizing agent

The compounds disclosed herein can be used as sterilizing agents, for example in high risk environments such as in hardware from hospitals or healthcare facilities. The World Health Organization (WHO) estimates that at any time, more than 1.4 million people worldwide are affected by infections acquired in hospitals. Cleaning, disinfection and sterilization saves lives and improves patient outcomes. Between 5% and 10% of patients admitted to modern hospitals in the developed world acquire one or more healthcare-associated infections. The Centers for Disease Control and Prevention (CDC) estimate that approximately 1.7 million healthcare-associated infections occur annually in hospitals in the United States, and are associated with nearly 100,000 deaths each year. Healthcare-associated infections are also an important problem in extended care facilities, including nursing homes and rehabilitation units. Transmission of healthcare-associated pathogens most frequently occurs via the hands of healthcare workers, who inadvertently contaminate their hands during various patient care activities. Less frequently, contaminated surfaces in healthcare facilities may contribute to the spread of healthcare-associated pathogens.

The varying levels of disinfection used in a healthcare facility may be defined by Spaulding's Classification (Sehulster, et al., Guidelines for environmental infection control in health-care facilities. Recommendations from CDC and the Healthcare Infection Control Practices Advisory Committee (HICPAC). Chicago Ill.; American Society for Healthcare Engineering/American Hospital Association; 2004). Spaulding's levels, non-critical, semi-critical, and critical, are based on the potential for infectious disease spread via equipment, instruments, and furniture as well as the level of sterility normally required for the body part coming in contact with it. Levels of disinfection that correlate with Spaulding's classification are low, intermediate, high, and sterilization. The US Centers for Disease Control (CDC) has further delineated disinfection levels for environmental surfaces in its “Guidelines for Environmental Infection Control in Health-Care Facilities”.

Critical items confer a high risk for infection if they are contaminated with any microorganism. Thus, objects that enter sterile tissue or the vascular system must be sterile because any microbial contamination could transmit disease. This category includes surgical instruments, cardiac and urinary catheters, implants, and ultrasound probes used in sterile body cavities. Semicritical items contact mucous membranes or nonintact skin. This category includes respiratory therapy and anesthesia equipment, some endoscopes, laryngoscope blades, esophageal manometry probes, cystoscopes, anorectal manometry catheters, and diaphragm fitting rings. These medical devices should be free from all microorganisms; however, small numbers of bacterial spores are permissible. Specific examples of critical or semi critical instruments include invasive endoscopes such as laparoscopes, and rigid instruments with no operating channel. Arthroscopes and laparoscopes which are inserted into sterile body cavities as well as accessory instrumentation should be sterile. Other examples include gastroscopes, duodenoscopes, sigmoidoscopes, proctoscopes, colonoscopes, bronchoscopes, and laryngoscopes.

3. Additional Methods of Use

Furthermore, it would be understood by those skilled in the art that the therapeutic methods described would not only apply to treatment in a subject, but could be applied to cell cultures, organs, tissues, or individual cells in vivo, ex vivo or in vitro.

### IV. Kits

Moreover, if a packaging material is utilized to package the pharmaceutical, compositions it may be biologically inert or lack bioactivity, such as plastic polymers, silicone, etc. and may be processed internally by the subject without affecting the effectiveness of the compound/antibiotic packaged and/or delivered therewith.

Additionally, the compounds and/or pharmaceutical compositions may be packaged with antibiotics as disclosed herein, preferred antibiotics of which include, but are not limited to, gentamicin, amikacin, kanamycin, neomycin, spectinomycin, neamine or any combination thereof.

Without further elaboration, it is believed that one skilled in the art can, using the preceding description, utilize the disclosed compounds, compositions, and methods to their fullest extent. The following examples are offered by way of illustration, not by way of limitation. While specific examples have been provided, the above description is illustrative and not restrictive. Any one or more of the features of the described forms can be combined in any manner with one or more features of any other described forms. Furthermore, many variations of the disclosed compounds, compositions, and methods will become apparent to those skilled in the art upon review of the specification.

The methods, compounds, and compositions herein described are further illustrated in the following examples, which are provided by way of illustration and are not intended to be limiting. It will be appreciated that variations in proportions and alternatives in elements of the components shown will be apparent to those skilled in the art and are within the scope of disclosed forms. Theoretical aspects are presented with the understanding that Applicants do not seek to be bound by the theory presented. All parts or amounts, unless otherwise specified, are by weight.

### EXAMPLES

### Materials and Methods

Bacterial strains: The Mu3 (ATCC 700698), USA300, COL, are methicillin-resistant strains of Staphylococcus aureus. The Newman, ATCC29213, RN4220, RN6390 are methicillin-sensitive strains. The AE-052, ST-45-502, ST-239-IIIA-503, ST-239-III-AH-504 are clinical isolates from Queen Mary Hospital, Hong Kong. Listeria monocytogenes (L. monocytogenes), Pseudomonas aeruginosa (P. aeruginosa) PA01, Streptococcus pneumoniae (S. pneumonia) R6 are reference strain, Burkholderia cepacia, Enterococcus faecalis, Escherichia coli (NDM-1) are multidrug resistant clinical isolates from Queen Mary Hospital, Hong Kong., Escherichia coli (IMP) is a laboratory strain [remove]. All the bacteria are culture in corresponding growth medium Brain Heart Infusion (BHI) Broth (OXOID), Mueller Hinton (MH) broth (BD), or Cation Adjusted Mueller Hinton (CAMH) broth (BD) at 37 ° C.

Drug libraries and chemicals: Structurally diverse small-molecule compound library is used for the high throughput screening. Small molecule compounds were purchased from Chembridge. Gentamicin (Sigma-Aldrich), kanamycin (Sigma-Aldrich), amikacin (Sigma-Aldrich), spectinomycin (Sigma-Aldrich), neomycin (Sigma-Aldrich), neamine (Sigma-Aldrich) are used in the minimum inhibitory concentration (MIC) test, high throughput screening, paper disc assays and checkerboard assay.

Minimal inhibitory concentration (MIC) test. Assays to determine the MIC of bacteria to the antibiotics are performed with a broth micro-dilution method in 96-wells microtitre plates with appropriate growth medium broths. For the determination of MIC of small-molecule compounds, the compounds are first subjected to two-fold serial dilution in DMSO, 1 μL of each diluent is added to the 96 well microtitre plates to give the desired concentration. All tests are performed in triplicates and repeated.

High throughput screening (HTS). Primary HTS screening is performed with customized robotic screening platform, fully automated Beckman Coulter Core system (Fullerton), integrated with liquid handler, washer, multidrop liquid dispenser (Thermo Scientific), Kendro robotics CO₂ incubator (Thermo Fisher Scientific), and DTX 880 multimode plate reader (Beckman coulter). The chemical library is pre-aliquoted into 384-well microtiter plates and upon screening, will give a final concentration of 20 μg/mL of compound in 50 μL BHI medium per well. All screening is done in 384-well polystyrene microtiter plates (Greiner Bio-One). Overnight culture of Mu3 is washed with BHI medium 3 times, diluted and plated to give 5×10⁵ CFU/mL per well. The plates are incubated at 37° C. for 16 hours and growth is monitored by optical density reading at 595 nm using an automated DTX 880 multimode detector. Screening using BHI medium is designated as the background screen. Potential chemical compounds showing synergistic properties with various antibiotics are screened using sub-MIC concentration antibiotic containing medium. Sub MIC concentration of antibiotics is selected for screening. BHI containing a final concentration for gentamicin 156.25 μg/mL is used correspondingly for individual experiments. Data analysis is done by custom designed data analysis software programmed by Jason Madar, Capilano University. Screening results with various antibiotics is normalized with the background screening to calculate the percentage of growth inhibition. Compounds with potential antibiotic activity enhancement effects are selected for secondary screening to validate the hits.

Selected compounds are prepared in different concentrations similar to the primary screening preparation. Identical experimental procedures are performed as primary screening procedures, except different concentrations of selected compound are tested. Compounds demonstrating bacterial growth inhibition in the presence of sub-MIC antibiotics are selected for more detailed characterization.

Checkerboard assay. The Checkerboard assay is performed in 96-well microtitre plates. Chemical compound MIC is first determined as previously described herein. The antibiotics are 2 fold serially diluted along the ordinate, the compounds are added along the abscissa to generate the checkerboard concentration gradients between the compounds and the antibiotics. Bacterial cultures are topped and incubated overnight. Bacterial growth is monitored by the OD absorbance at 595 nm.

Cytotoxicity assay. MTT assays are performed for selected compounds on Vero cells according to product instructions.

Paper disc diffusion assay. The paper disc diffusion assays are performed by impregnating 10 μL of 5 mM compounds or appropriate amount of antibiotics in to the 8 mm diameter filter paper disc. The discs are then placed on a uniform swabbed lawn of bacteria on BHI agar and grown overnight at 37° C. The larger zone of inhibition correlates with smaller MIC.

Paper disc diffusion and lux assays. A single colony of bioluminescent S. aureus carrying the hla-promoter lux reporting system was resuspended in 200 μl of sterile water, diluted 1000-fold into 0.7% (w/v) BHI agar and overlaid onto BHI agar plates. 10 μl of the 50 mM analogues compounds dissolved in DMSO were impregnated to paper disc and placed on the lawn of bacteria. The plates were incubated overnight at 37° C. The luminescence was detected with Xenogen IVIS 100 in vivo imaging system (Xenogen, Alameda, Calif.).

Compound selectivity optimization. The lead compound from the primary screens is selected and analogues of the compound are searched and ordered for better performing compounds. The MIC and MTT for the analogues are determined. The selective index is calculated for each compound by comparing the compound toxicity versus the compound efficacy.

In vivo experiments. All animal experiments were carried out following the Laboratory Animal Unit (LAU) of the University of Hong Kong's regulation. 6-8 weeks old female BALB/c mice were infected with 4×10⁸ CFU of S. aureus . Compound 3 or injection buffer control (PBS with 5% DMSO and 2% Tween 80), were pretreated one day before infection, two dosages were given per day post infection. 3 days after infection, the animals were euthanized; kidneys, livers and spleens were homogenized in PBS, and plated on BHI agar for quantitative bacterial culture.

Results

Preliminary testing of the MIC value of three antibiotics (Gentamicin, Ceftriaxone, and Levofloxacin) against MRSA Mu3 was performed according to microbroth dilution methods. This was to establish the reference for the appropriate amount of antibiotics to be used for HTS for hit identification of compounds that could have synergistic activities or potentiate the antibiotic's antimicrobial effect. The MIC value of Gentamicin against MRSA Mu3 was determined to be 1000 μg/mL. Adjusted sub MIC concentration of antibiotics was used for the screenings; a final concentration for gentamicin 156.25 μg/mL was used for individual screening experiment.

High throughput screening of the structurally diverse small molecule library at a final concentration of 20 μg/mL was carried out against the Mu3 strain in plain BHI. Growth was monitored by the OD readings. This screening was to initiate the background antimicrobial activities of each compound against the Mu3 strain. This was later compared against the individual screenings containing the sub MIC concentration of antibiotic. Antibiotic potentiating compounds ideally have no background antimicrobial activities in the background screen and exhibit growth inhibition in the presence of sub MIC concentration of antibiotic. Primary hit compounds were isolate through the initial screenings after the normalization of the sub MIC screens with the background screen. The selected compounds were subjected to the secondary screening to refine and validate the combinatory effect of the small molecules with the antibiotic.

Among which Compound 13 was finalized for more in depth studies. This compound shows no sign of MIC, up to an undetermined concentration, which the compound solubility limits the higher concentrations to be tested.

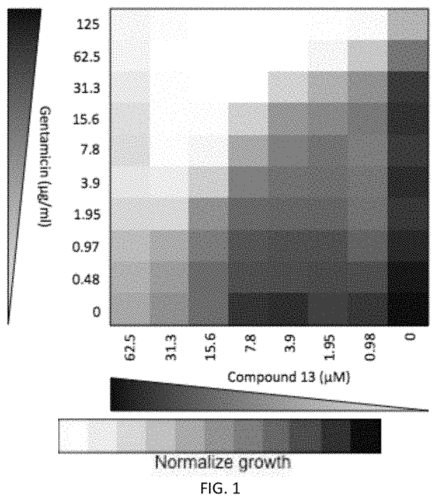

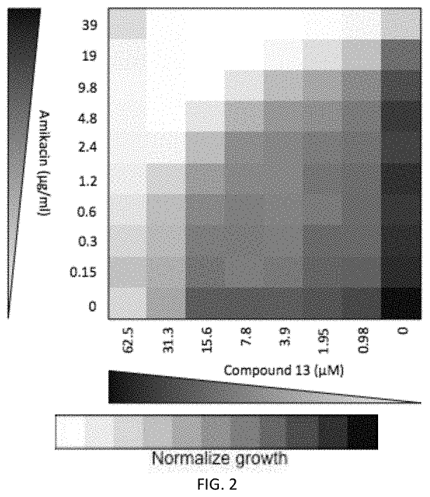

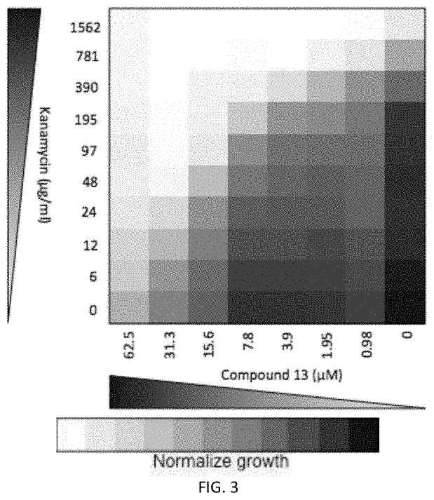

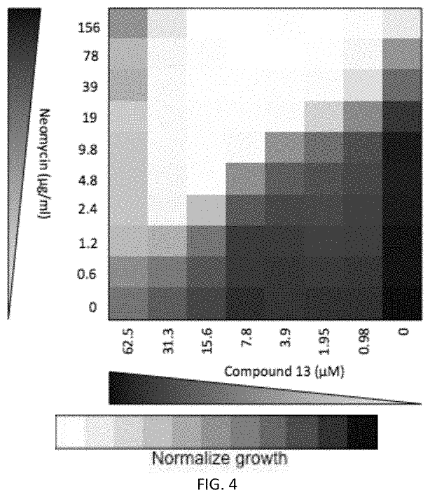

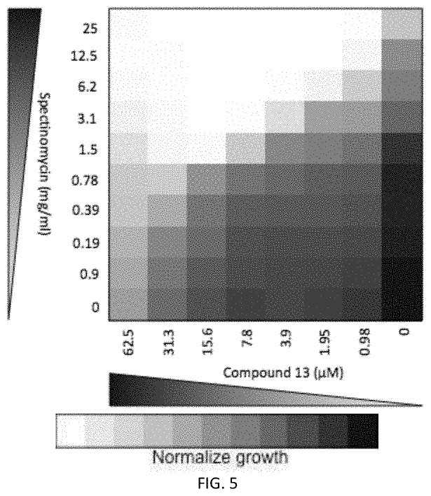

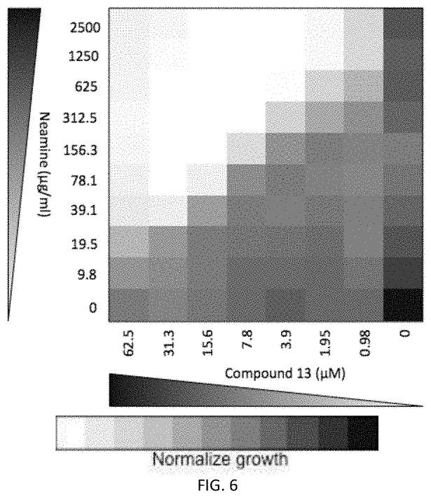

Checkerboard assays were performed with the lead compound, compound 13 , against gentamicin (FIG. 1) and other aminoglycosides, amikacin (FIG. 2), kanamycin (FIG. 3), neomycin (FIG. 4), spectinomycin (FIG. 5), neamine (6). All of the antibiotics tested can be potentiated by compound 13 and show a combinatory improvement.

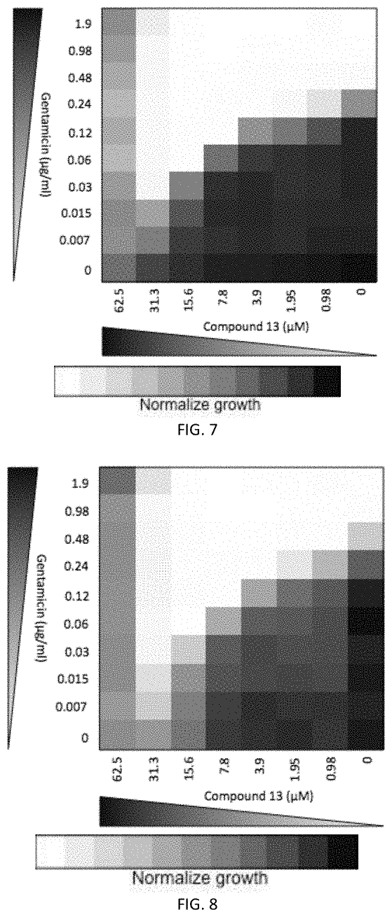

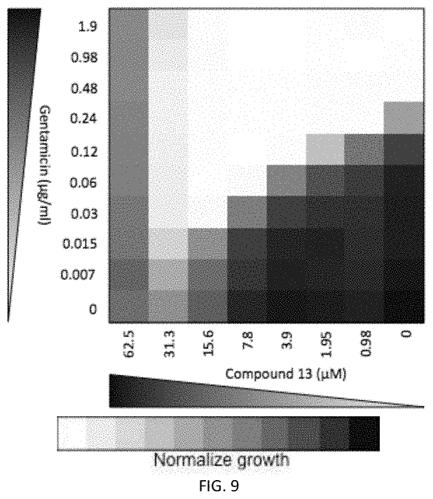

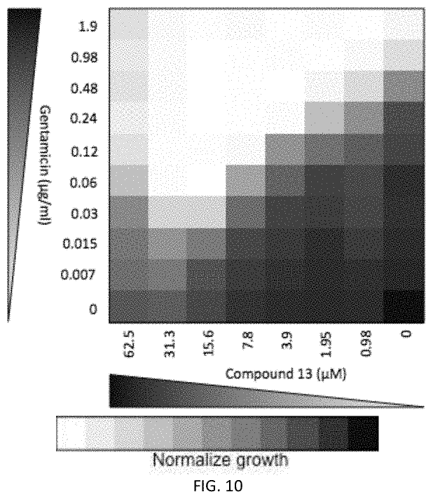

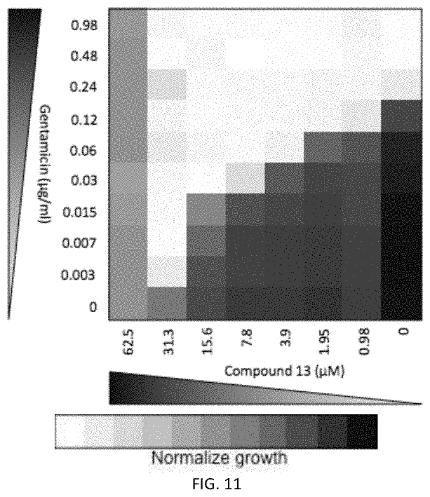

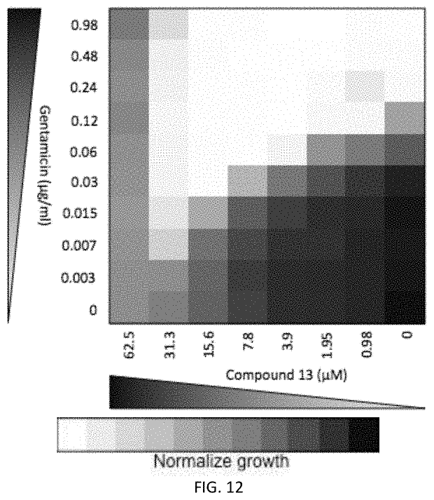

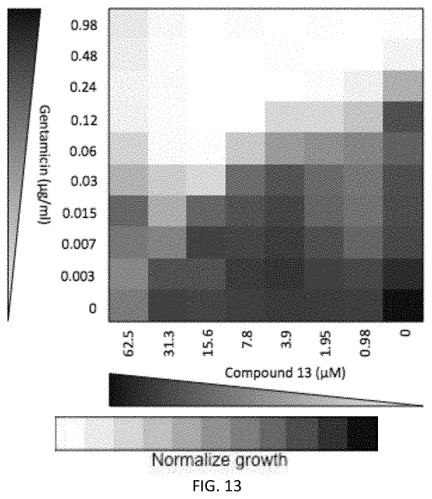

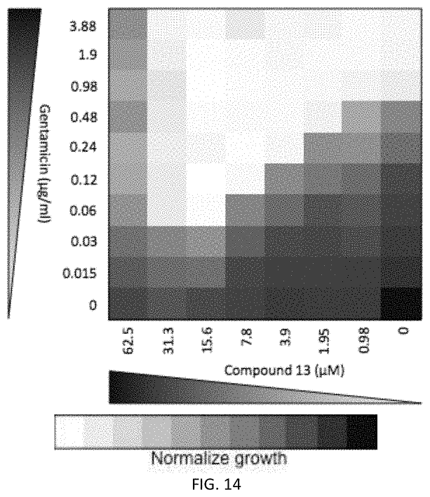

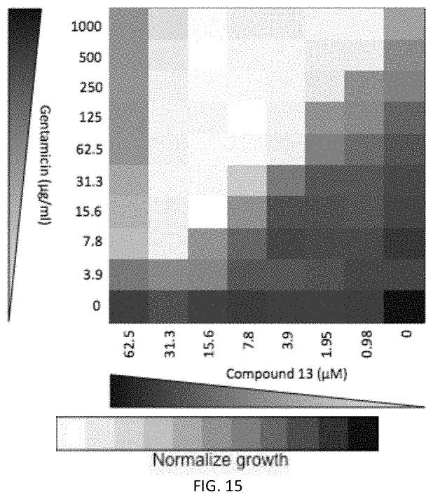

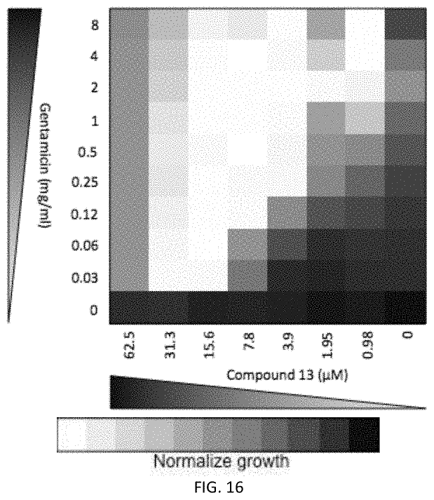

compound 13 can also extend its combinatory effect with gentamicin in other Staphylococcus aureus (S. aureus ) strains. The effects could be seen with gentamicin sensitive strains such as ATCC 29213 (FIG. 7), Newman (FIG. 8), RN4220 (FIG. 9), USA300 (FIG. 10), COL (FIG. 11), RN6390 (FIG. 12), ST-45-502 (FIGS. 13) and AE-052 (FIG. 14), as well as well as in gentamicin resistant strains such as ST-239-III-AH-504 (FIG. 15), ST-239-III-AH-503 (FIG. 16).

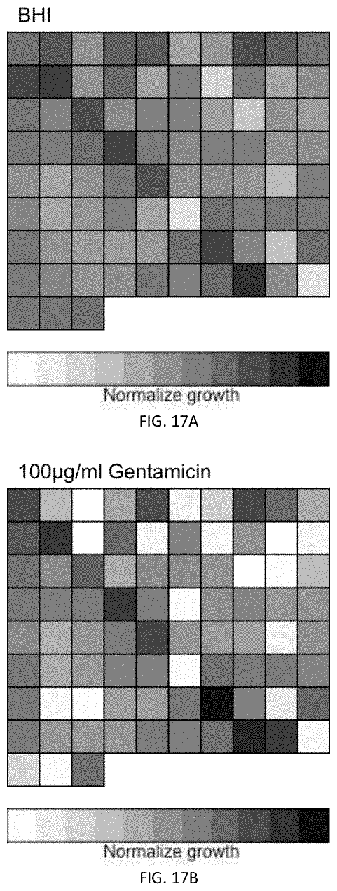

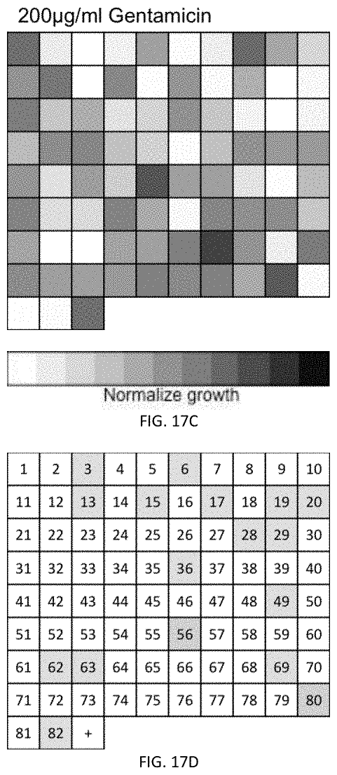

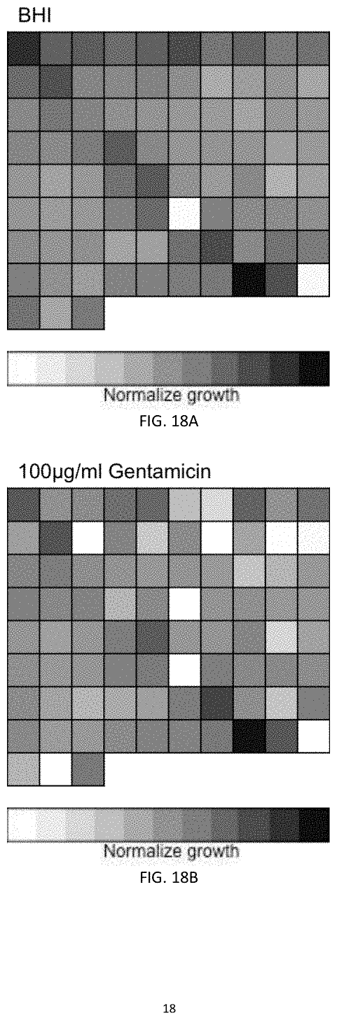

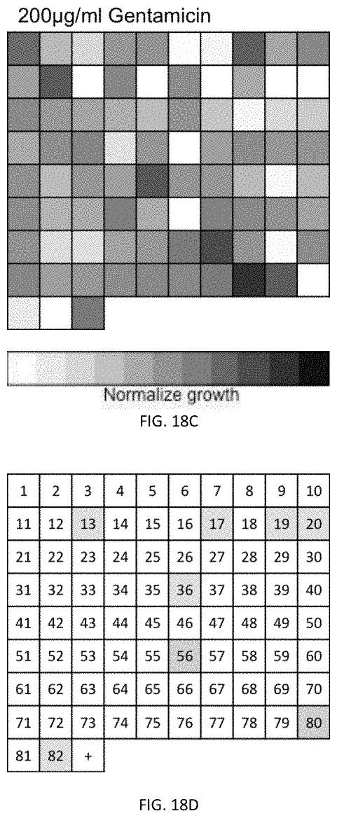

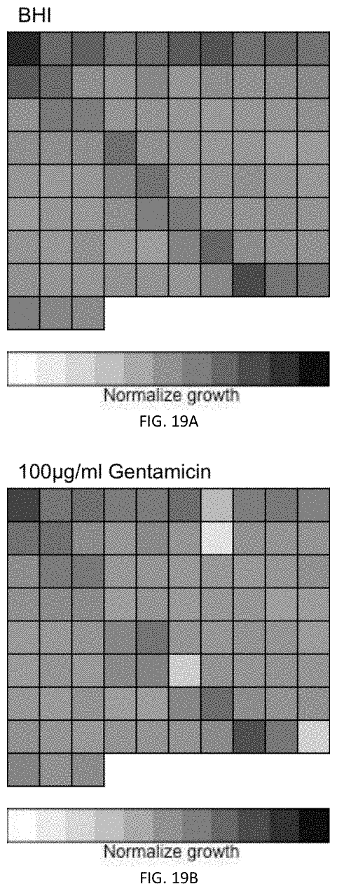

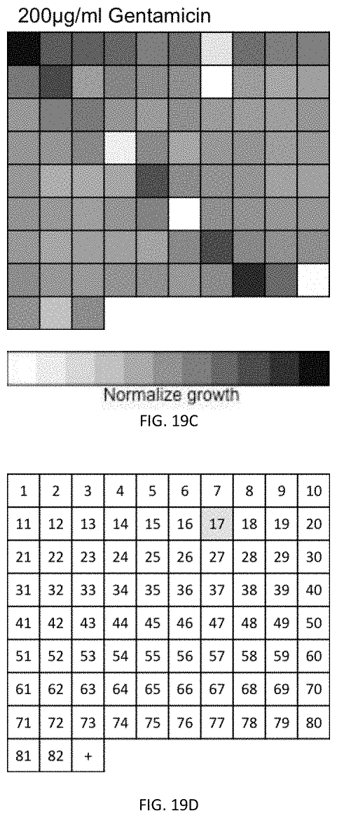

Compound 13 demonstrated its aminoglycoside potentiating properties in S. aureus , structural analogues of compound 13 were purchased for lead compound optimization. Secondary like screening was performed with different concentrations of each analogue compounds were tested with different gentamicin concentration, 50 μM, 20 μM and 5 μM compound concentration were used against, BHI containing, 0 μg/ml, 100 μg/ml and 200 μg/ml gentamicin. Analogues showing activities in the screenings were selected (FIG. 17, 18, 19), summarized in Table 1.

TABLE 1
Summarizing the numerical relative growth of the analogues secondary screening
at various concentrations with various concentration of gentamicin.

| | 5 μM | | | 20 μM | | | 50 μM | | | |
|---|---|---|---|---|---|---|---|---|---|---|
| [Cpd] | BHI | 100 ug/ | 200 ug/ | BHI | 100 ug/ | 200 ug/ | BHI | 100 ug/ | 200 ug/ | Virulence |
| [Genta] | only | ml | ml | only | ml | ml | only | ml | ml | hla promoter |
| 1 | 1.38 | 1.29 | 1.45 | 1.22 | 1.1 | 1.05 | 1 | 1.11 | 1.01 | − |
| 2 | 1.15 | 1.1 | 1.2 | 1.08 | 0.93 | 0.65 | 1.07 | 0.62 | *0.24 | + |
| 3 | 1.18 | 1.12 | 1.18 | 1.09 | 0.94 | 0.4 | 0.88 | *0.09 | *0.08 | −−− |
| 4 | 1.1 | 1.07 | 1.16 | 1.05 | 1.04 | 0.89 | 1.05 | 0.76 | 0.2 | − |
| 5 | 1.13 | 1.08 | 1.06 | 1.08 | 1.06 | 0.92 | 1.07 | 1.1 | 0.8 | − |
| 6 | 1.2 | 1.13 | 1.11 | 1.14 | 0.61 | *0.1 | 0.79 | *0.2 | *0.11 | − |
| 7 | 1.22 | 0.67 | *0.34 | 1.02 | 0.4 | 0.15 | 0.87 | 0.45 | *0.23 | − |
| 8 | 1.12 | 1.06 | 1.13 | 1.07 | 1.07 | 1.09 | 1.1 | 1.12 | 1.02 | −− |
| 9 | 1.15 | 1.09 | 1.06 | 1 | 0.92 | 0.78 | 1.06 | 1.02 | 0.76 | + |
| 10 | 1.11 | 1.04 | 1.06 | 1.03 | 1.03 | 0.96 | 1 | 0.73 | 0.41 | + |
| 11 | 1.2 | 1.12 | 1.09 | 1.04 | 0.84 | 0.82 | 1.12 | 1.07 | 0.89 | + |
| 12 | 1.14 | 1.12 | 1.27 | 1.12 | 1.11 | 1.1 | 1.14 | 1.16 | 0.98 | + |
| 13 | 0.99 | 1 | 0.89 | 0.97 | *0.09 | *0.09 | 0.88 | *0.09 | *0.09 | − |
| 14 | 0.95 | 0.94 | 1.03 | 0.95 | 0.97 | 0.96 | 1.02 | 1.04 | 0.93 | ++ |
| 15 | 1.02 | 1.01 | 1 | 0.97 | 0.52 | *0.09 | 0.79 | *0.18 | *0.11 | + |
| 16 | 0.94 | 0.95 | 0.99 | 0.94 | 0.95 | 0.94 | 0.96 | 0.95 | 0.88 | ++ |
| 17 | 0.99 | *0.35 | *0.1 | 0.74 | *0.09 | *0.09 | 0.43 | *0.14 | *0.13 | − |
| 18 | 0.98 | 0.97 | 0.9 | 0.85 | 0.8 | 0.76 | 0.97 | 0.88 | 0.7 | − |
| 19 | 0.99 | 0.96 | 0.83 | 0.91 | *0.1 | *0.09 | 0.76 | *0.09 | *0.08 | −− |
| 20 | 1.01 | 0.96 | 0.88 | 0.78 | *0.14 | *0.09 | 0.91 | *0.17 | *0.14 | − |
| 21 | 0.97 | 1 | 0.94 | 0.96 | 0.96 | 0.95 | 0.99 | 1 | 0.96 | + |
| 22 | 1.08 | 1.08 | 1.05 | 1.01 | 0.99 | 0.88 | 0.94 | 0.91 | 0.52 | − |
| 23 | 1.07 | 1.09 | 1.08 | 0.97 | 0.94 | 0.79 | 1.1 | 1.05 | 0.71 | ++ |
| 24 | 0.96 | 0.94 | 0.95 | 0.93 | 0.92 | 0.73 | 0.92 | 0.72 | *0.32 | ++ |
| 25 | 0.96 | 0.95 | 0.92 | 0.91 | 0.89 | 0.62 | 0.95 | 0.91 | 0.42 | ++ |
| 26 | 0.94 | 0.94 | 0.99 | 0.89 | 0.91 | 0.92 | 0.95 | 0.91 | 0.9 | +++ |
| 27 | 0.92 | 0.93 | 0.91 | 0.87 | 0.88 | 0.55 | 0.81 | 0.88 | 0.53 | ++ |
| 28 | 0.94 | 0.93 | 0.91 | 0.81 | 0.56 | *0.14 | 0.51 | *0.09 | *0.19 | − |
| 29 | 0.96 | 0.94 | 0.89 | 0.9 | 0.67 | *0.39 | 0.89 | *0.11 | *0.08 | + |
| 30 | 0.98 | 0.98 | 0.95 | 0.88 | 0.89 | 0.51 | 0.83 | 0.6 | *0.18 | −− |
| 31 | 0.99 | 0.98 | 0.95 | 0.96 | 0.98 | 0.77 | 0.98 | 0.98 | 0.61 | −− |
| 32 | 0.99 | 1 | 0.96 | 0.95 | 0.96 | 0.93 | 0.97 | 0.96 | 0.91 | + |
| 33 | 0.99 | 1 | 1.02 | 1 | 0.97 | 0.97 | 1.01 | 0.97 | 0.93 | + |
| 34 | 1.12 | 0.9 | *0.24 | 1.09 | 0.67 | *0.33 | 1.13 | 1.16 | 0.57 | + |
| 35 | 0.98 | 0.96 | 1.01 | 0.96 | 0.95 | 0.9 | 0.97 | 0.96 | 0.45 | +++ |
| 36 | 0.95 | 0.92 | 0.83 | 0.9 | *0.09 | *0.09 | 0.92 | *0.11 | *0.11 | +++ |
| 37 | 0.93 | 0.94 | 0.94 | 0.92 | 0.91 | 0.83 | 0.96 | 0.9 | 0.57 | − |
| 38 | 0.93 | 0.95 | 0.96 | 0.91 | 0.93 | 0.94 | 0.96 | 0.94 | 0.9 | + |
| 39 | 0.91 | 0.89 | 0.9 | 0.82 | 0.89 | 0.9 | 0.89 | 0.86 | 0.85 | ++ |
| 40 | 0.91 | 0.91 | 0.92 | 0.88 | 0.89 | 0.91 | 0.91 | 0.89 | 0.85 | + |
| 41 | 0.94 | 0.92 | 0.93 | 0.9 | 0.9 | 0.93 | 0.9 | 0.92 | 0.87 | − |
| DMSO | 1 | 1 | 1 | 1 | 1 | 1 | 1 | 1 | 1 | +++ |
| 42 | 0.93 | 0.93 | 0.78 | 0.81 | 0.82 | 0.63 | 0.77 | 0.7 | *0.33 | + |
| 43 | 0.94 | 0.96 | 0.8 | 0.88 | 0.89 | 0.9 | 0.89 | 0.87 | 0.82 | ++ |
| 44 | 1.03 | 1.02 | 0.83 | 1.03 | 0.99 | 0.83 | 0.98 | 0.97 | 0.47 | ++ |
| 45 | 1.11 | 1.11 | 1.24 | 1.1 | 1.09 | 1.11 | 1.09 | 1.12 | 1.08 | +++ |
| 46 | 0.98 | 0.95 | 1 | 0.93 | 0.9 | 0.9 | 0.9 | 0.87 | 0.81 | +++ |
| 47 | 0.96 | 0.94 | 0.98 | 0.87 | 0.91 | 0.89 | 0.91 | 0.85 | 0.8 | +++ |
| 48 | 0.98 | 0.97 | 0.96 | 0.95 | 0.97 | 0.64 | 0.87 | 0.8 | *0.31 | −−− |
| 49 | 0.95 | 0.95 | 0.86 | 0.7 | 0.42 | *0.13 | 0.61 | *0.2 | *0.1 | −−− |
| 50 | 0.93 | 0.91 | 0.87 | 0.82 | 0.82 | 0.64 | 0.96 | 0.9 | 0.6 | ++ |
| 51 | 0.91 | 0.92 | 0.96 | 0.9 | 0.93 | 0.94 | 0.93 | 0.99 | 0.94 | − |
| 52 | 0.95 | 0.96 | 0.93 | 0.86 | 0.89 | 0.69 | 0.76 | 0.74 | *0.36 | − |
| 53 | 0.93 | 0.92 | 0.89 | 0.89 | 0.9 | 0.76 | 0.88 | 0.84 | *0.38 | + |
| 54 | 0.99 | 1.01 | 0.96 | 0.97 | 0.98 | 0.99 | 0.96 | 0.95 | 0.94 | ++ |
| 55 | 1.05 | 1.04 | 1.04 | 1.05 | 1 | 0.74 | 0.77 | 0.95 | 0.73 | −− |
| 56 | 1.08 | 0.52 | **0.1 | **0.11 | **0.09 | **0.09 | **0.32 | **0.12 | **0.14 | + |
| 57 | 0.97 | 0.96 | 0.99 | 0.98 | 0.99 | 0.98 | 1 | 1.01 | 0.93 | +++ |
| 58 | 0.96 | 0.95 | 0.97 | 0.95 | 0.95 | 0.95 | 0.97 | 0.98 | 0.9 | +++ |
| 59 | 0.98 | 0.96 | 0.95 | 0.94 | 0.95 | 0.92 | 0.98 | 0.97 | 0.91 | +++ |
| 60 | 0.96 | 0.95 | 0.95 | 0.93 | 0.94 | 0.81 | 0.98 | 0.94 | 0.52 | ++ |
| 61 | 0.96 | 0.94 | 0.93 | 0.94 | 0.94 | 0.93 | 0.98 | 0.98 | 0.77 | +++ |
| 62 | 0.93 | 0.92 | 0.83 | 0.91 | 0.8 | *0.37 | 0.88 | *0.14 | *0.09 | +++ |
| 63 | 0.97 | 0.97 | 0.88 | 0.93 | 0.67 | *0.35 | 0.84 | *0.11 | *0.08 | + |
| 64 | 0.91 | 0.89 | 0.87 | 0.8 | 0.76 | 0.81 | 0.83 | 0.81 | 0.78 | +++ |
| 65 | 0.9 | 0.9 | 0.85 | 0.82 | 0.83 | 0.89 | 0.86 | 0.82 | 0.8 | +++ |
| 66 | 1.04 | 1.02 | 1.02 | 1.03 | 0.99 | 1 | 1.01 | 0.98 | 0.95 | + |
| 67 | 1.16 | 1.14 | 1.26 | 1.14 | 1.16 | 1.14 | 1.13 | 1.25 | 1.13 | ++ |
| 68 | 1 | 1 | 1.02 | 0.95 | 0.94 | 0.92 | 0.94 | 0.95 | 0.88 | +++ |
| 69 | 0.97 | 0.97 | 0.98 | 1.02 | 0.56 | *0.11 | 0.55 | *0.27 | *0.23 | − |
| 70 | 0.97 | 0.97 | 0.99 | 1 | 0.98 | 0.94 | 1.02 | 1.04 | 0.97 | +++ |
| 71 | 0.97 | 0.98 | 1 | 0.98 | 0.95 | 0.96 | 0.97 | 0.97 | 0.92 | ++ |
| 72 | 0.95 | 0.95 | 0.94 | 0.93 | 0.86 | 0.84 | 0.93 | 0.85 | 0.81 | +++ |
| 73 | 0.94 | 0.93 | 0.92 | 0.85 | 0.88 | 0.9 | 0.87 | 0.85 | 0.81 | +++ |
| 74 | 0.97 | 0.96 | 0.97 | 0.97 | 0.95 | 0.94 | 0.91 | 0.85 | 0.85 | ++ |
| 75 | 0.96 | 0.96 | 0.98 | 0.98 | 0.98 | 0.95 | 0.99 | 0.97 | 0.95 | +++ |
| 76 | 0.95 | 0.96 | 0.93 | 1 | 0.97 | 0.95 | 0.95 | 0.95 | 0.92 | ++ |
| 77 | 1.02 | 1.01 | 1.01 | 1.01 | 1 | 1 | 1.02 | 1 | 0.95 | − |
| 78 | 1.26 | 1.24 | 1.38 | 1.28 | 1.28 | 1.21 | 1.18 | 1.21 | 0.74 | + |
| 79 | 1.1 | 1.09 | 1.14 | 1.13 | 1.11 | 1.09 | 0.91 | 1.15 | 1.07 | ++ |
| 80 | 1.1 | 0.49 | **0.12 | **0.16 | **0.09 | **0.09 | **0.33 | **0.15 | **0.13 | − |
| 81 | 1.05 | 1.03 | 0.98 | 1.03 | 0.68 | *0.24 | 0.98 | 0.4 | *0.1 | − |
| 82 | 1.03 | 0.98 | 0.61 | 0.78 | *0.09 | *0.09 | 0.99 | *0.13 | *0.11 | − |
| DMSO | 1 | 1 | 1 | 1 | 1 | 1 | 1 | 1 | 1 | +++ |

*indicates the relative growth smaller than 40%,
**indicate compound oriented growth inhibition.
+/− indicates induction or repression on hla promoter.

Structure and function analysis was carried out to narrow down the functional core structure (Table 2, 3, 4, 5).

TABLE 2
Structure function analysis of compounds with over 30% growth inhibitions
under 50 μM compound concentration and 200 μg/ml gentamicin

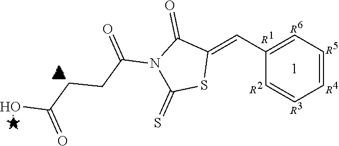

| | | 50 μM + | 20 μM+ |
|---|---|---|---|
| | | 200 μg/ml | 200 μg/ml |
| | | Gentamicin | Gentamicin |
| | | Growth | Growth |
| Compound | | inhibition | inhibition |
| No. | Side Chain | Percentage | Percentage |
| 2 | -Ring1 R4 addition 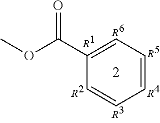 -Ring2 R4 addition —Cl | 76 | 35 |
| 4 | -Ring1 R4 addition 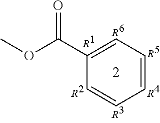 -Ring2 R2 addition —Cl -Ring2 R4 addition —Cl | 80 | 11 |
| 10 | -Ring1 R4 addition 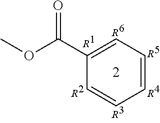 | 59 | 4 |
| 28 | -Ring1 R2 addition 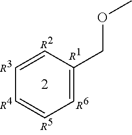 -Ring2 R4 addition —Cl | 81 | 86 |
| 29 | -Ring1 R4 addition 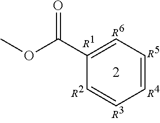 -Ring2 R5 addition —CH₃ | 92 | 61 |
| 30 | -Ring1 R4 addition 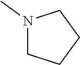 -Ring1 R5 addition —F | 82 | 49 |
| 31 | - Ringl R4 addition 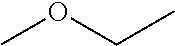 | 39 | 23 |
| 35 | -Ring1 R4 addition 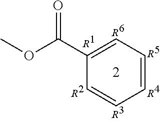 -Ring2 R4 addition —F | 55 | 10 |
| 36 | -Ring1 R4 addition 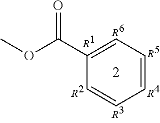 -Ring2 R4 addition 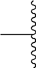 | 89 | 91 |
| 37 | -▴ addition —CH₃ -Ring1 R4 addition 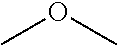 | 43 | 17 |
| 48 | -Ring1 R4 addition 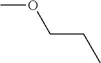 | 69 | 36 |
| 49 | -Ring1 R4 addition 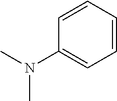 | 90 | 87 |
| 52 | -Ring1 R2 addition 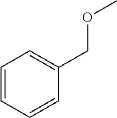 | 64 | 31 |
| 53 | -▴ addition | 62 | 24 |
| | —CH₃ | | |
| | -Ring1 R4 addition | | |
| | —Cl | | |
| 56 | -Ring1 R2 addition 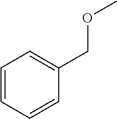 -Ring1 R5 addition —Br | 86 | 91 |
| 60 | -Ring2 R5 addition 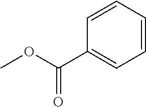 | 48 | 19 |
| 62 | -Ring1 R5 addition 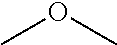 -Ring1 R4 addition 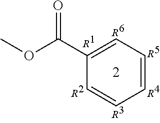 -Ring2 R3 addition —CH₃ | 91 | 63 |
| 80 | -Ring1 R2 addition 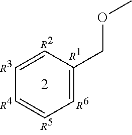 -Ring1 R5 addition —Br -Ring2 R6 addition —Cl | 87 | 91 |

Table 2 shows a structure function analysis of selected analogues with over 30% growth inhibitions with compound concentration of 50 μM and 200 μg/ml gentamicin. The analysis shows that some functional groups improve the combinatory growth inhibition effect and retains growth inhibition of S. aureus Mu3 at a lower gentamicin concentration.

TABLE 3
Structure function analysis of compounds with over 30% growth inhibitions
under 50 μM compound concentration and 200 μg/ml gentamicin

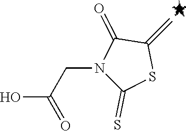

| | | 50 μM + | 20 μM + |
|---|---|---|---|
| | | 200 g/ml | 200 g/ml |
| | | Gentamicin | Gentamicin |
| | | Growth | Growth |
| Compound | | inhibition | inhibition |
| No. | Side Chain | Percentage | Percentage |
| 42 | -★ addition 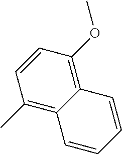 | 67 | 37 |
| 81 | -★ addition 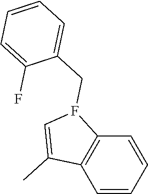 | 90 | 76 |

Table 3 shows a structure function analysis of selected analogues with over 30% growth inhibitions with compound concentration of 50 μM and 200 μg/ml gentamicin. The analysis shows that some functional groups improve the combinatory growth inhibition effect and retains growth inhibition of S. aureus Mu3 at a lower gentamicin concentration.

TABLE 4
Structure function analysis of compounds with over 30% growth
inhibitions under 50 μM compound concentration and 200 μg/ml
gentamicin

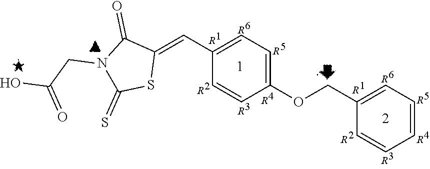

| | | 50 μM + | 20 μM+ |
|---|---|---|---|
| | | 200 μg/ml | 200 μg/ml |
| | | Gentamicin | Gentamicin |
| | | Growth | Growth |
| Compound | | inhibition | inhibition |
| No. | Side Chain | Percentage | Percentage |
| 3 | [symbol] | 92 | 60 |
| 6 | -Ring1 R5 addition 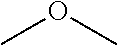 -Ring2 R4 addition —Cl | 89 | 90 |
| 7 | - [symbol] replaced with 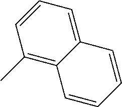 | 77 | 85 |
| 20 | -Ring1 R5 addition 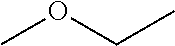 -Ring2 R4 addition —Cl | 86 | 91 |
| 22 | -Ring1 R5 addition 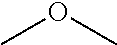 | 48 | 12 |
| 25 | -Ring1 R5 addition 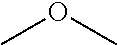 -Ring2 R2 addition —F | 58 | 38 |
| 34 | -Ring1 R5 addition 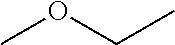 -Ring2 R4 addition —Cl -Ring2 R5 addition —Cl | 43 | 67 |
| 44 | -Ring1 R5 addition 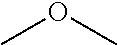 -Ring2 R4 addition —Br | 53 | 17 |
| 50 | Ring1 R5 addition 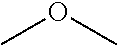 -Ring2 R2 addition —Cl -Ring2 R4 addition —F | 40 | 36 |
| 69 | -★ ester-linkage | 77 | 89 |
| 82 | -Ring2 R4 addition | 89 | 91 |
| | —Cl | | |

Table 4 shows a structure function analysis of selected analogues with over 30% growth inhibitions with compound concentration of 50 μM and 200 μg/ml gentamicin. The analysis shows that some functional groups improve the combinatory growth inhibition effect and retains growth inhibition of S. aureus Mu3 at a lower gentamicin concentration.

TABLE 5
Structure function analysis of compounds with over 30% growth ii hibitions
under 50 μM compound concentration and 200 μg/ml gentamicin

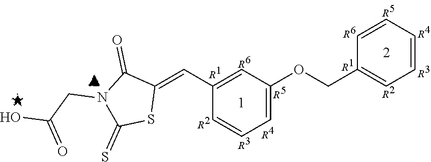

| | | 50 μM + | 20 μM + |
|---|---|---|---|
| | | 200 μg/ml | 200 μg/ml |
| | | Gentamicin | Gentamicin |
| | | Growth | Growth |
| Compound | | inhibition | inhibition |
| No. | Side Chain | Percentage | Percentage |
| 13 | -Ring2 R4 addition | 91 | 91 |
| | —Cl | | |
| 15 | -Ring 1 R4 addition 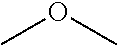 -Ring2 R4 addition —Cl | 89 | 91 |
| 17 | -Ring2 R4 addition | 87 | 91 |
| | —Cl | | |
| | -Ring2 R6 addition | | |
| | —Cl | | |
| 18 | -Ring 1 R4 addition 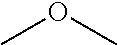 -Ring2 R4 addition —Cl -Ring2 R6 addition —Cl | 30 | 24 |
| 19 | -Ring2 R6 addition | 92 | 91 |
| | —Cl | | |
| 24 | -Ring1 R4 addition 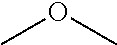 | 68 | 27 |
| 27 | -Ring1 R4 addition 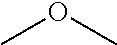 -Ring2 R6 addition —F | 47 | 45 |
| 63 | -Ring2 R6 addition | 92 | 65 |
| | —F | | |

Table 5 shows a structure function analysis of selected analogues with over 30% growth inhibitions with compound concentration of 50 μM and 200 μg/ml gentamicin. The analysis shows that some functional groups improve the combinatory growth inhibition effect and retains growth inhibition of S. aureus Mu3 at a lower gentamicin concentration.

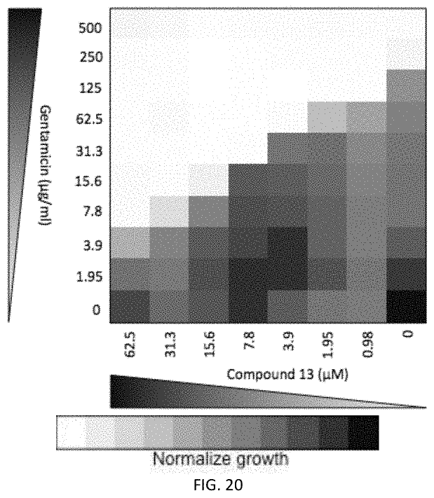

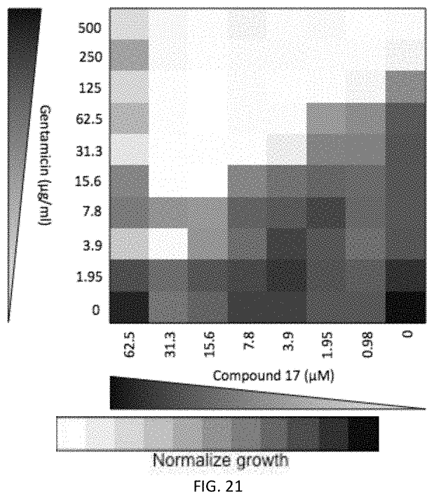

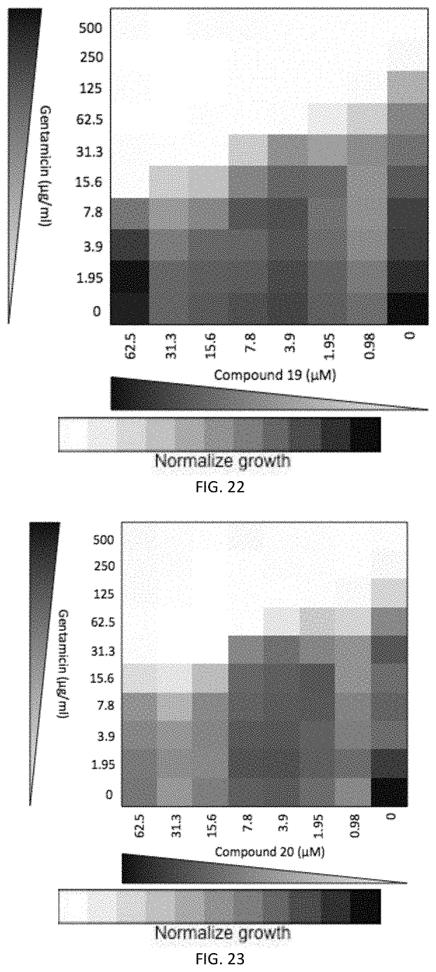

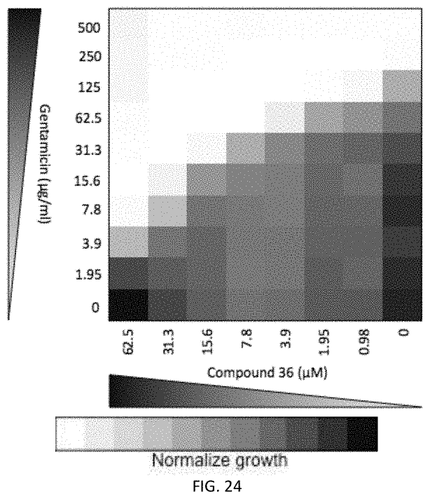

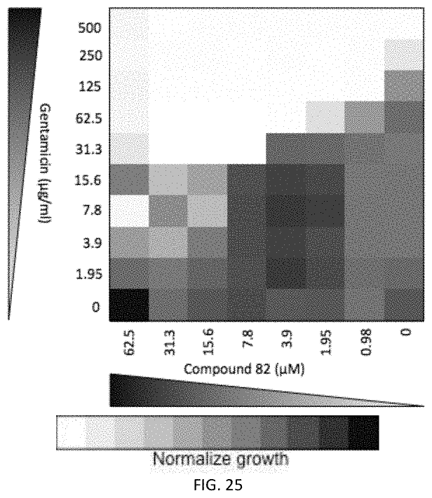

Paper disc assays were used for direct assess of the potentiating effect of the selected compounds with potent activities. Checkerboard assays were carried out for the selected compounds, compound 13 (FIG. 20), compound 17 (FIG. 21), compound 19 (FIG. 22), compound 20 (FIG. 23), compound 36 (FIG. 24) and compound 82 (FIG. 25). The cytotoxicity of the 6 compounds was tested with MTT assays in Vero cells, results were summarized in Table 6.

TABLE 6
Summary table of selected compound and their attributes.

| | Effective at <20 μM | Combinatory | |
|---|---|---|---|
| | with 100 μM | effect with | |
| Compound | gentamicin | gentamicin | Cytotoxicity |
| 2 | | | |
| 3 | | | Low |
| 4 | | | |
| 6 | + | | |
| 7 | +++ | | |
| 13 | +++++ | Potentiating | High |
| 15 | ++ | | |
| 17 | +++++ | Potentiating | High |
| 19 | +++++ | Potentiating | Medium |
| 20 | +++++ | Potentiating | Medium/Low |
| 24 | | | |
| 28 | ++ | | |
| 29 | + | | |
| 30 | | | |
| 36 | +++++ | Potentiating | High |
| 42 | | | |
| 48 | | | |
| 49 | +++ | | |
| 52 | | | |
| 53 | | | |
| 56 | NA | | |
| 62 | | | |
| 63 | + | | |
| 69 | ++ | | |
| 80 | NA | | |
| 81 | ++ | | |
| 82 | +++++ | Potentiating | Low |

Paper disc diffusion and lux assays of the tested analogues compounds reveal the anti-virulence properties of some analogues, summarized on Table 1. Testing of other analogues of compound 3 identified some potential virulence factor inhibitor, Table 3.

TABLE 7
Relative lux signal of selected compounds on the
hla promoter, each compound is used at 25 μM.

| Compound No. | MIC (μM) | Relative lux signal |
|---|---|---|
| 3 | 500 | 0.00439 |
| 83 | 500 | 0.83975 |
| 84 | 500 | 0.7261 |
| 85 | 500 | 0.80121 |
| 86 | 500 | 0.6369 |
| 87 | 500 | 0.2657 |
| 88 | 500 | 0.97768 |
| 89 | 500 | 0.9655 |
| 90 | 500 | 1.1926 |
| 91 | 500 | 0.97015 |
| 92 | 500 | 0.83975 |

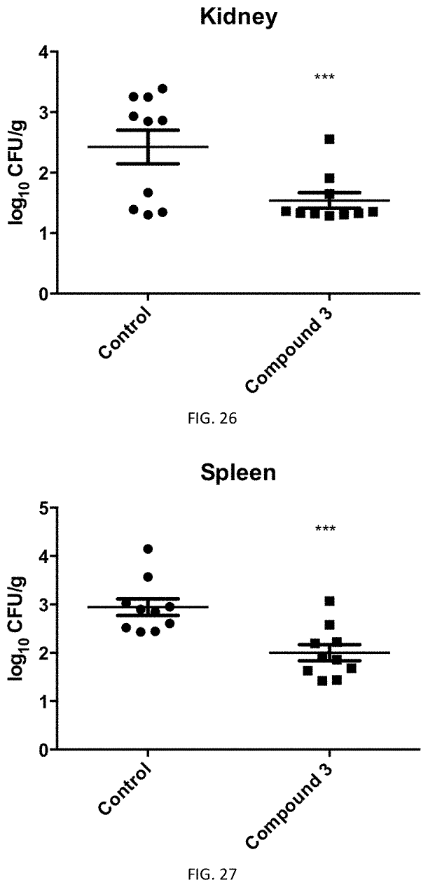

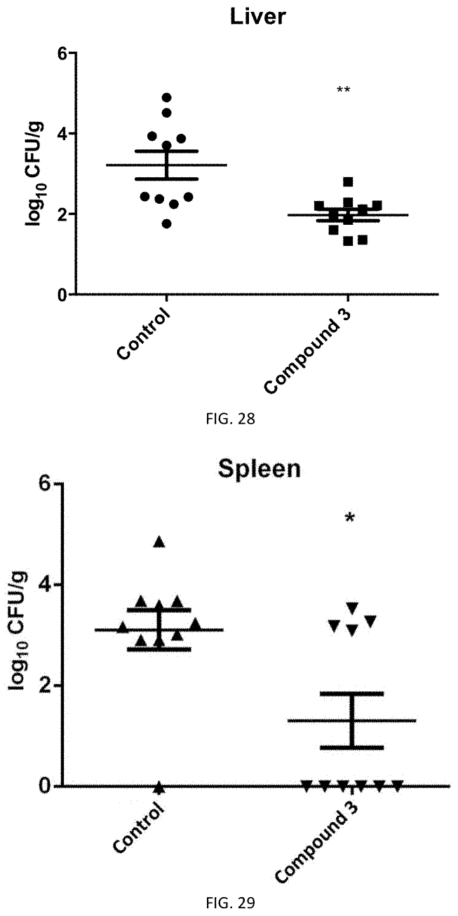

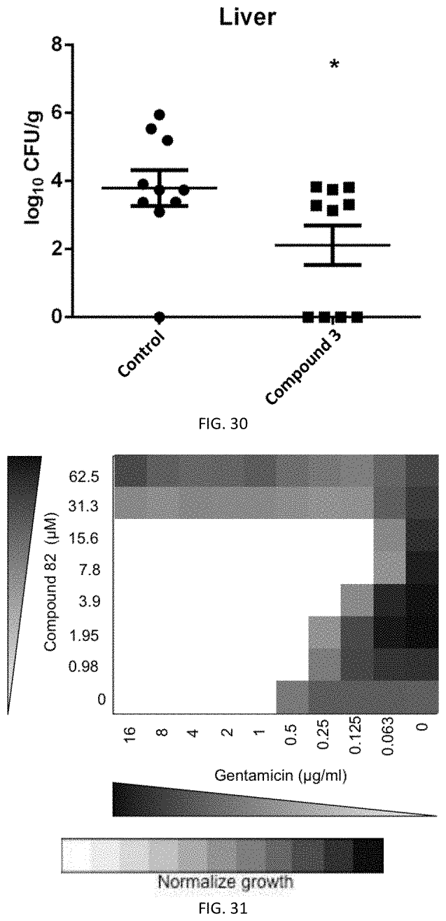

The selected compound 3 as treatment showed reduction of kidney (FIG. 26), spleen (FIG. 27), and liver (FIG. 28) bacterial load in BALB/c mice infection model with Mu3 strains. Similar results were observed in BALB/c mice infection model with USA300 strains, reduction in spleen (FIG. 29), and liver (FIG. 30) bacterial load.

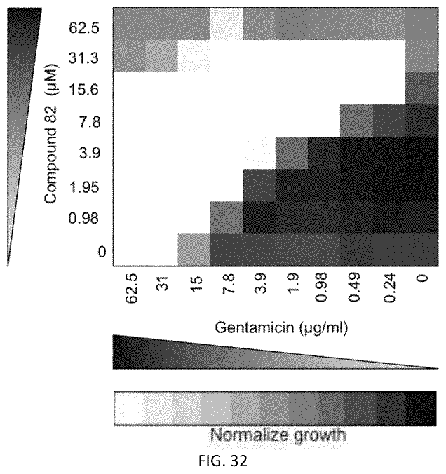

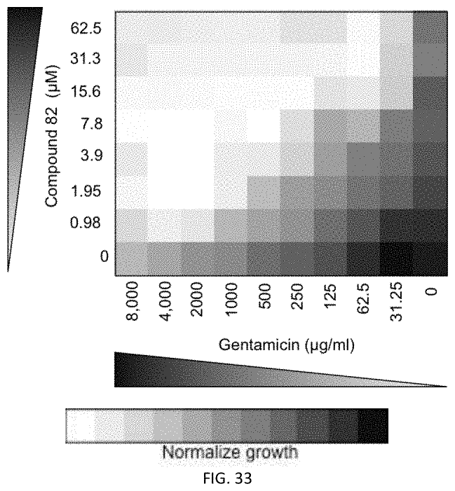

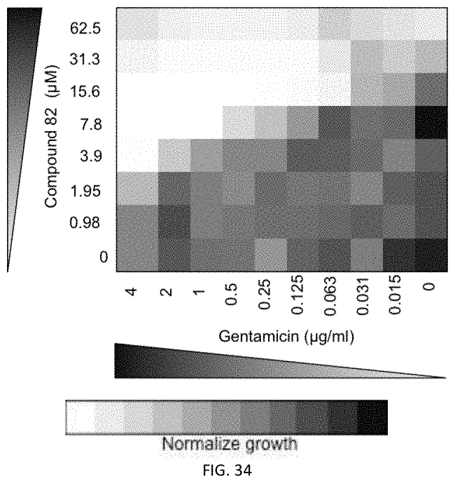

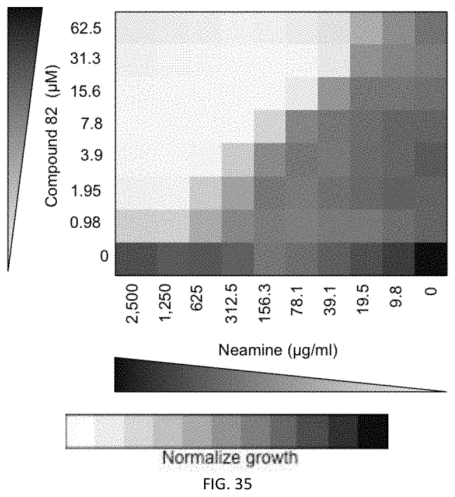

Activities of the selected compound 82 on other bacteria were tested. Checkerboard assays were conducted to evaluate the activities for other Gram positive bacteria, L. monocytogenes (FIG. 31), E. faecalis (FIG. 32), and S. pneumoniae (FIG. 34). A gentamicin potentiating effect in the presence of compound 82 was observed in, L. monocytogenes, E. faecalis and S. pneumoniae. Furthermore, compound 82 also demonstrated the synergistic effect with Gram negative bacteria, E. coli (FIG. 33). compound 82 can also mediate virulence suppression on other Gram negative bacteria, by repressing the pigment formation in P. aeruginosa and B. cepacia. Finally, compound 82 (FIG. 35) retained its potentiating effect against S. aureus in the presence of neamine.

Discussion

Previously, through a high-throughput screening platform, Applicants successfully isolated a small molecule, nucleozin, which targets the influenza A nucleoprotein (Kao et al., Nature biotechnology, 28(6):600-605 (2010)). The same library of structurally diverse small molecule compounds was screened. By performing the background screening (screening with BHI medium, without antibiotics) at a final concentration of 20 μg/mL, small molecule compounds with direct bacterial inhibitions were excluded from the screening. Screenings with the presence of sub MIC antibiotics allow the identification of small molecules with a potential bacterial growth inhibition effect in combination with antibiotics. The top 10% of compounds showing a combinatorial effect with antibiotics were selected for further studies. Dual concentration secondary screens were conducted; compounds were added at a final concentration of 100 μm/mL and 10 μg/mL. This method refined the screening result by reducing false positives from the primary screening, as well as fortifying the reproducibility of success.

The top compound from the secondary screen was subjected to further analysis. MIC of the compound (compound 13 ) in Mu3 was first characterized. The concentration tested range was from 500 μM to 0.98 μM. MICs higher than 500 μM were not determined. Followed by the MIC determination, the checkerboard assay of compound 13 against different aminoglycoside was performed to assess the aminoglycoside combinatory effect. The Fractional inhibitory concentration (FIC) index was normally used to assess the combinatory effect (Meletiadis et al., Antimicrobial agents and chemotherapy, 54(2):602-609 (2010)). FIC index of less than or equal to 0.5 is considered as synergistic, but for compound's MIC higher than 500 μM, which the actual MIC cannot be determined, are considered to be potentiating if the antibiotic concentration required for growth inhibition is decreased by at least 4 fold antibiotic MIC.

Through sequential screening processes, Applicants were able to narrow down a chemical compound library to a lead compound for future chemical optimization for antibiotics potentiator. As shown, compound 13 demonstrated the potentiating effect in combination with aminoglycoside, gentamicin, kanamycin, amikacin, neomycin and spectinomycin, which are commonly used in clinical settings. Furthermore, at compound concentration of around 50 μM, it can reduce the antibiotic MIC below the corresponding clinical breakpoint, making an ineffective drug usable again for MRSA treatment. Neamine is represents the core structure common to all aminoglycosides being used in the clinical settings. Furthermore, neamine derivative libraries were constructed for novel aminoglycosides antibiotics screening. compound 13 showed potentiating effect with neamine further indicated that the potentiating effect is a broad spectrum characteristic among the aminoglycoside family.

Applicant validated that other strains of S. aureus are exhibiting similar observation of antibiotic potentiating effect by compound 13 and gentamicin. Regardless of gentamicin sensitivity, all tested strains demonstrated the enhanced susceptibility to gentamicin in the presence of compound 13. This provides a useful antibiotic cocktail treatment for S. aureus infection.

Applicants identified several potent analogue compounds providing potentiating effect in combinatorial use with gentamicin in the lead compound optimization screening. Compound 13, 17, 36 showed potentiating activities for gentamicin, but is accompany by high cytotoxicity from Vero cell MTT assays. Compound 19, 20 were found to have less cytotoxicity while retaining promising potentiating effect with gentamicin. Finally compound 82 is found to exhibit low cytotoxicity as well as maintaining good level of potentiating effect with gentamicin against S. aureus .

Applicants expanded the effective host range of the compound 82 to other tested species of bacteria, including Gram positive L. monocytogenes, E. faecalis and S. pneumoniae, Gram negative bacteria, E. coli. Applicants also demonstrated that compound 82 could elicit virulence suppression on other Gram negative bacteria, by repressing the pigment formation in P. aeruginosa and B. cepacia, which are important for colonization in host.

Applicants also identified apart from combinatory effect with current antibiotics as a potential therapeutic option, some of the compounds by themselves can repress virulence activities. Compound 3, 48, 49 shows significant repression in hla promoter lux reporter assay. Applicants have demonstrated the concept of using compound 3 as anti-virulence can reduce organs' bacterial loads in In vivo mice model, indicating the compounds analogues themselves can be a therapeutic method for bacterial infection. Analogues of Compound 3 like Compound 87 also demonstrate the hla promoter lux repression, suggesting the anti-virulence property of the compound.

Applicants demonstrate the success of using HTS to isolate small molecule compound having potentiating effect with aminoglycoside against bacteria. The family of compound could also by themselves act as anti-virulence agent in reducing infectivity of bacteria. The compounds are useful for downstream chemical optimizations to increase the combinatory effect with antibiotics and enhance the anti-virulence property, while providing a reduction in cytotoxicity. The biological activities of the disclosed compounds are novel, and they provide commercial applications for potentiating the activities of commonly used antibiotics along with anti-virulence use.

While in the foregoing specification the disclosed compounds, compositions, and methods have been described in relation to certain forms thereof, and many details have been put forth for the purpose of illustration, it will be apparent to those skilled in the art that the disclosed compounds, compositions, and methods are susceptible to additional forms and that certain of the details described herein can be varied considerably without departing from the basic principles of the invention.

All references cited herein are incorporated by reference in their entirety. The disclosed compounds, compositions, and methods can be embodied in other specific forms without departing from the spirit or essential attributes thereof and, accordingly, reference should be made to the appended claims, rather than to the foregoing specification, as indicating the scope of the disclosed compounds, compositions, and methods.

1. A composition for inhibiting dihydrofolate reductase in a microorganism including a compound represented by Formula I 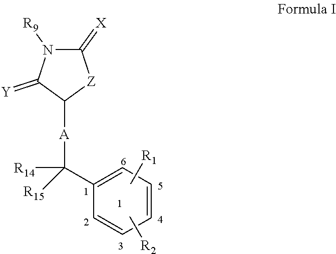
wherein X and Y are independently O, S, or NR_{10;}
wherein Z is O, S, CR₁₁R_{12,} or NR_{13;}
wherein R_{1,} R_{2,} R_{9,} R_{10,} R_{11,} R_{12,} R_{13,} and R_{15,} are independently absent, hydrogen, —C(O)R_{18,} —C(W)NRi₉R_{20,} or substituted or unsubstituted alkyl, alkenyl, alkynyl, alkoxy, cycloalkyl, heterocyclyl, cycloalkenyl, heterocycloalkenyl, aryl, heteroaryl, aroxy, arylalkyl, heteroalkyl, alkylaryl, halogen, alkylheteroaryl, —NR₁₆R_{17;}
wherein R₁₄ is independently absent, —C(W)NR₁₉R_{20,} or substituted or unsubstituted alkyl, alkenyl, alkynyl, alkoxy, cycloalkyl, heterocyclyl, cycloalkenyl, heterocycloalkenyl, aryl, heteroaryl, aroxy, arylalkyl, heteroalkyl, alkylaryl, halogen, alkylheteroaryl, —NR₁₆R_{17;}
wherein R₁₆ and R₁₇ are independently hydrogen, substituted or unsubstituted alkyl, alkenyl, alkynyl, cycloalkyl, heterocyclyl, cycloalkenyl, heterocycloalkenyl, aryl, heteroaryl, arylalkyl, heteroalkyl, alkylaryl, or alkylheteroaryl;
wherein R₁₈ is hydrogen, hydroxyl, or substituted or unsubstituted alkyl, alkenyl, alkynyl, cycloalkyl, alkoxy, heterocyclyl, cycloalkenyl, heterocycloalkenyl, aryl, aroxy, heteroaryl, arylalkyl, heteroalkyl, alkylaryl, or alkylheteroaryl;
wherein W is O, S, or NR_{21;}
wherein R_{19,} R_{20,} and R₂₁ are independently hydrogen or substituted or unsubstituted alkyl, alkenyl, alkynyl, cycloalkyl, heterocyclyl, cycloalkenyl, heterocycloalkenyl, aryl, heteroaryl, arylalkyl, heteroalkyl, alkylaryl, or alkylheteroaryl;
wherein A is a double bond or single bond; and
wherein R₁₅ is absent when A is a double bond,

2. The composition of claim 1, wherein the compound is 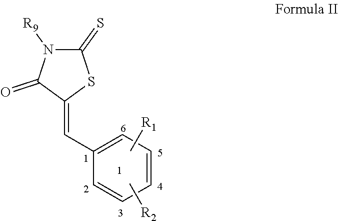
wherein R₁ is —O—R_{4,} —NR₅R_{8,} or —CH₂—R_{5;} R₂ is halogen, absent, —O—CH_{3,} or —O—CH₂—CH₃; R₉ is —COOH, —CH₂—COOH, —CH₂—O—CH_{3,} or —CH₂—CH₂—O—CH_{3;} R₄ is —CH₂—R ₅ or —CO—R_{5;} R₅ is 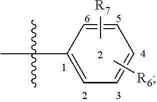
R₆ is a halogen or absent; and R₇ is halogen or absent.

3. The composition of claim 2, wherein R₁ is at a position selected from the group consisting of 2, 3, 4, 5, and 6; R₆ is at position 4; R₇ is at position 2 or 6 and R₂ is at position 3, 4, or 5.

4. The composition of claim 2 wherein the compound is 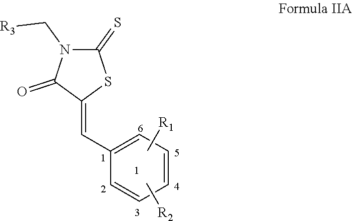
wherein R₁ is —O—R_{4,} —NR₅R_{8,} or —CH₂—R_{5;} R₂ is halogen, absent, —O—CH_{3,} or —O—CH₂—CH₃; R₃ is —COOH or —CH₂—COOH; R₄ is —CH₂—R₅ or —CO—R_{5;} R₅ is 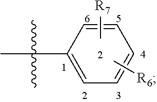
R₆ is halogen or absent; and R₇ is halogen or absent.

5. The composition of claim 4, wherein R₁ is at a position selected from the group consisting of 2, 3, 4, 5, and 6; R₆ is at position 4; R₇ is at position 2 or 6; and R₂ is at position 3, 4, or 5.

6. The composition of claim 1, wherein the compound is selected from the group consisting of compounds 2-4, 6-7, 10, 13, 15, 17-20, 22, 24-25, 27-31, 34-37, 42, 44, 48-50, 52-53, 56, 60, 62-63, 69, 80-82, and 87.

7. The composition of claim 6, further comprising an antibiotic.

8. The composition of claim 7, wherein the antibiotic is selected from the group consisting of gentamicin, amikacin, kanamycin, neomycin, spectinomycin, neamine and combinations thereof.
